(19) United States
(12) Patent Application Publication
(10) Pub. No.: US 2024/0279313 A1
(43) Pub. Date: Aug. 22, 2024

(54) BARRIER FUNCTION PRESERVING PEPTIDES FOR MEMBRANES

(71) Applicant: The Regents of the University of California, Oakland, CA (US)

(72) Inventors: Michelle Lee, Santa Barbara, CA (US); Wujing Xian, Los Angeles, CA (US); Nathan W. Schmidt, Santa Monica, CA (US); Gerard C. L. Wong, Los Angeles, CA (US)

(73) Assignee: The Regents of the University of California, Oakland, CA (US)

(21) Appl. No.: 18/292,207

(22) PCT Filed: Aug. 11, 2022
(86) PCT No.: PCT/US22/40072

§ 371 Date: Jan. 25, 2024

Related U.S. Application Data
(60) Provisional application No. 63/231,941, filed on Aug. 11, 2021.

(51) Int. Cl. C07K 14/775; A61K 38/00; G16B 15/20

(52) U.S. Cl. CPC C07K 14/775; G16B 15/20; A61K 38/00

(57) ABSTRACT

The invention disclosed herein comprises peptides designed to include an amino acid sequence that confers such peptides with an ability to induce positive Gaussian curvature (and/or an ability to inhibit negative Gaussian curvature) in mammalian cell membranes. The invention disclosed herein further includes methods of using such peptides, for example as a non-hormonal contraceptive, an antiviral agent, an anti-inflammatory agent, or a therapeutic agent which can, for example, reduce neurotransmitter or hormone secretion in order to treat neuropsychiatric or endocrine disorders.

### CROSS-REFERENCE TO RELATED APPLICATIONS

This application claims the benefit under 35 U.S.C. Section 119(e) of co-pending and commonly-assigned U.S. Provisional Patent Application Ser. No. 63/231,941, filed on Aug. 11, 2021, and entitled “BARRIER FUNCTION PRESERVING PEPTIDES FOR MEMBRANES” which application is incorporated by reference herein.

### STATEMENT REGARDING FEDERALLY SPONSORED RESEARCH AND DEVELOPMENT

This invention was made with government support under Grant Number 1808459, awarded by the National Science Foundation. The government has certain rights in the invention.

### TECHNICAL FIELD

The present invention relates to peptides having selected functional properties and methods for making and using them.

### BACKGROUND OF THE INVENTION

Biological membranes undergo changes in morphology during a variety of cellular processes that require membrane remodeling. This phenomenon occurs in membrane fusion, the merging of two membranes into one, such as that observed between: gamete membranes during fertilization, between viral and host cell membranes during infections from enveloped viruses, between synaptic vesicles and cell membranes of presynaptic neurons during the exocytic release of neurotransmitters. Other forms of membrane remodeling include membrane fission and membrane pore formation. Membrane fission, the division of one membrane into two, proceeds through similar configurational steps as membrane fusion but in the reverse order. Membrane fission occurs during the budding and scission of newly assembled virions from host cells, as well as endocytic processes. Membrane pore formation, the creation of a channel in the bilayer that bridges the two sides of the membrane, compromises the membrane integrity and increases permeability. During cell lysis, the formation of pores causes the cell plasma membrane to destabilize and break down, resulting in the loss of membrane barrier function and eventual cell death. While diverse, all of these described membrane events involve the generation of membrane curvature.

Previous work has identified the specific type of membrane curvature involved in and required for these biological membrane restructuring processes as negative Gaussian curvature (31, 59-61). Evidence further indicates that the generation of negative Gaussian curvature is a common root mechanism for membrane restructuring in general (61-63). However, artisans have never published or otherwise disclosed that it is possible to design specific compositions of matter that can negate or cancel this type of curvature on membranes.

There is a need in the art for peptides having an ability to induce positive Gaussian curvature (and/or an ability to inhibit negative Gaussian curvature) of cell membranes, and methods for making and using such peptides.

### SUMMARY OF THE INVENTION

As disclosed herein, we have discovered how to make and use peptides that can induce positive Gaussian curvature (PGC, dome-shaped surface curvature) in cell membranes. Using a combination of synchrotron SAXS measurements, mean-field theory, in vitro cell studies and in vivo animal studies, we show that such peptides can inhibit NGC (saddle-shaped surface curvature) and its consequences, including membrane permeation, lytic cell death, and associated organ damage from inflammation. As discussed below, such peptides can be used to modulate cell membrane physiology in a number of clinically relevant contexts.

The invention disclosed herein has a number of embodiments. Embodiments of the invention include, for example, compositions of matter comprising one or more peptides selected for its ability to induce positive Gaussian curvature in a mammalian cell. Such peptide compositions of the invention can be made by any one of a variety of methods for making such molecules (e.g., naturally/expressed or chemically synthesized peptides). Typically in such compositions, the peptide amino acid sequence is selected to comprise three distinct faces/sectors as an amphipathic α-helix when visualized in a helical wheel plot, (e.g. as shown in FIG. 10). Amphipathicity (also called amphiphilicity) describes the spatial clustering of hydrophobic (nonpolar) and hydrophilic (polar) amino acid residues on two opposite faces (sides) oriented along the axis of the helix. Helical wheel plots are commonly used to illustrate the residue arrangement and amphipathicity of helices. A helical wheel plot is a two-dimensional representation of the projection down the axis of a helix, with the residues drawn around a unit circle. Because an α-helix consists of 3.6 residues per turn, each residue is offset from the preceding one by 100°. Each distinct face can be described by the angle subtended by its corresponding sector of residues, (e.g., in FIG. 10, the angle subtended by the nonpolar face is indicated by θ and its span is delineated by the dotted lines). The three sectors of the invention are described as: a nonpolar face, a polar face, and positively charged face such that: (1) The nonpolar face consists of 6-11 adjacent residues on the helical wheel plot, which together subtend a radial angle (perpendicular to the axis of the helix) of 120-220°; and the nonpolar face contains 4 or more residues that are classified as nonpolar amino acids, which are defined here as being any of the following: A, C, F, G, I, L, M, P, V, W, Y; (2) The polar face consists of 4-13 adjacent residues on the helical wheel plot, which together subtend a radial angle (perpendicular to the axis of the helix) of 80-260°; and the polar face contains 4 or more residues that are classified as polar amino acids, which are defined here as being any of the following: A, C, D, E, G, H, K, N, P, Q, R, S, T, Y; and (3) the positively charged face consists of 1-6 adjacent residues on the helical wheel plot, which together subtend a radial angle (perpendicular to the axis of the helix) of 20-120°; and the positively charged face contains 1 or more residues that are classified as positively charged amino acids, which are defined here as being any of the following: H, K, R. In some embodiments of the invention, such peptides must contain at least one of the following residues: G, N, Q, S. In some embodiments of the invention, such peptides do not contain more than 5 aromatic nonpolar residues (F, W, Y). In certain embodiments of the invention, such peptides exhibit a net charge between −3 and +3 at physiological pH.

In certain embodiments of the invention, concentrations of one or more peptides in the composition are selected to be sufficient to induce positive Gaussian curvature in a mammalian cell contacted with the composition; and/or concentrations of one or more peptides in the composition are selected to be sufficient to counteract negative Gaussian curvature in a mammalian cell contacted with the composition. In illustrative embodiments of the invention, a peptide is selected or designed such that a peptide:lipid molar ratio of 1/10 or lower can inhibit the generation of NGC magnitudes |<K>| that are 1×10⁻³ nm² or greater in lipid membranes composed of 20/80 1,2-dioleoyl-sn-glycero-3-phospho-L-serine (CAS number: 90693-88-2)/1,2-dioleoyl-sn-glycero-3-phosphoethanolamine (CAS number: 4004-05-1).

In nonlimiting illustrative embodiments of the invention, a peptide comprises the sequence: G-D-A-V-R-E-W-F-E-K-A-W-Q-R-V-R-E-F-F (“AP1” SEQ ID NO: 1), FFERVRQWAKEFWERVADG (“AP2” SEQ ID NO: 7), GDAVREVIEKAVQRVREIV (“AP3” SEQ ID NO: 8), GDAVKEWFEKAWQKVKEFF (“AP4” SEQ ID NO: 9), GERAKEWVEAFWEKAREYF (“AP5” SEQ ID NO: 10), GERVKEFFEAFFEKAREYW (“AP6” SEQ ID NO: 11), GEKAKEWVQAFWQKAKEYF (“AP7” SEQ ID NO: 12), GEKVKEFFQAFFQKAKEYW (“AP8” SEQ ID NO: 13), GDAVKEWFEKAWQKVKEFL (“AP9” SEQ ID NO: 14), GEQLKQKFQEFWDKLKEWY (“AP10” SEQ ID NO: 15), GEKLKQKAQEFFDAVKEWF (“AP11” SEQ ID NO: 16), GKEKAEEFFQALKEWFDKFKN (“AP12” SEQ ID NO: 17) or GEQFKQAFQEWWDKLKEKY (“AP13” SEQ ID NO: 18). In some embodiments of the invention, a peptide of the invention is further coupled to an agent/compound such as a plurality of amino acids, for example a polypeptide sequence encoded in a mammalian genome (e.g., a human protein or portion thereof). Typically, peptide compositions of the invention also include a pharmaceutically acceptable carrier. In some embodiments of the invention, peptide(s) having an ability to induce positive Gaussian curvature in a mammalian cell is/are chemically synthesized. In other embodiments of the invention, peptide(s) having an ability to induce positive Gaussian curvature in a mammalian cell is/are expressed by a cell comprising a polynucleotide encoding the peptide(s).

Embodiments of the invention include compositions of matter comprising a polynucleotide encoding a peptide selected for its ability to induce positive Gaussian curvature in a mammalian cell. In one nonlimiting illustrative embodiment of the invention, the polynucleotide encodes the sequence: G-D-A-V-R-E-W-F-E-K-A-W-Q-R-V-R-E-F-F (SEQ ID NO: 1). In certain embodiments of the invention, the polynucleotide encoding the peptide is fused in frame to a polynucleotide sequence encoding a plurality of amino acids (e.g., a human protein or a segment of a human protein). In some embodiment of the invention, the polynucleotide encoding the peptide is disposed within an expression vector comprising sequences for expressing the peptide in a mammalian cell. In certain embodiments of the invention, the vector is disposed within a bacterial, yeast or mammalian cell.

Embodiments of the invention include methods of using the peptides disclosed herein. Small angle X-ray scattering measurements indicate that peptides of the invention exhibit inhibitory effects against the induction of negative Gaussian curvature on membranes by directly interacting with the lipid membrane or interacting with key membrane proteins including those involved in sperm-egg fusion, viral entry, and proinflammatory cell lysis. In vitro assays have further shown such peptides to inhibit cells from infection by enveloped viruses, which is consistent with X-ray findings. In this context, embodiments of the invention include, for example, methods of inducing positive Gaussian curvature in a mammalian cell and/or counteracting negative Gaussian curvature in a mammalian cell. These methods comprise contacting the mammalian cell with a composition comprising a peptide disclosed herein: wherein concentrations of such peptides in the composition are selected to be sufficient to induce positive Gaussian curvature in the mammalian cell and/or counteract negative Gaussian curvature in the mammalian cell when the mammalian cell is contacted with the composition. In some embodiments of the invention, these methods are adapted to stabilize natural or artificial lipid membranes so as to inhibit said membrane permeabilization. In other embodiments of the invention, these methods are adapted to prevent fertilization by inhibiting membrane fusion between sperm and egg cells. In other embodiments of the invention, these methods are adapted to inhibit viral replication processes requiring negative Gaussian membrane curvature (e.g., processes comprising viral entry into host cells and/or the release of virions from host cells). In other embodiments of the invention, these methods are adapted to mitigate virus-associated inflammation. In other embodiments of the invention, these methods are adapted to mitigate inflammation resulting from lytic cell death or tissue damage by inhibiting permeabilization of cell membranes. In other embodiments of the invention, these methods are adapted to interfere with and/or inhibit membrane fusion required for secretory processes, including, but not limited to, neurotransmitter release, hormone secretion, and enzyme release.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. LPS-induced endotoxemia associates with multi-organ neutrophil infiltration, NET release, and tissue damage. FIG. 1(a-g) C57BL6/J mice were challenged with 10 mg/kg of LPS (E. coli O111:B4) or vehicle (PBS) for 24 hours. (a) Schematic representation of endotoxemia model. FIG. 1(b, c) From left to right, liver, lung, kidney and heart. FIG. 1(b) Representative immunofluorescence micrographs showing tissue-infiltrated neutrophils (Ly6G, green) and nuclei (DAPI, blue) in indicated organs. Scale bar: 50 μm. FIG. 1(c) Displayed is the quantification of infiltrated neutrophils in sections from indicated organs of PBS or LPS-treated mice. n=4-5 mice/group. FIG. 1(d) Representative immunofluorescence micrographs showing tissue NET-like structures (indicated with an asterisk) defined with the markers Ly6G, myeloperoxidase (MPO), histone H4, and DNA (DAPI), and extranuclear histone H4 in indicated organs. Extranuclear histone H4 is displayed as a binary image obtained from the subtraction of DAPI signal from histone H4 signal. Scale bar: 50 μm. Displayed is the quantification of NETs (upper panel) and extranuclear histone H4 area (lower panel) in liver FIG. 1(e) and lung FIG. 1(f) in sections from indicated organs of PBS or LPS-treated mice. n=4-5 mice/group. FIG. 1(g) Representative immunofluorescence micrographs showing dead cells (TUNEL, red) and nuclei (DAPI, blue). Scale bar: 50 μm. Correlogram showing Pearson correlations between tissue TUNEL⁺ cells, neutrophil and NET numbers, and extranuclear histone H4 area in liver (upper panel) and lung (lower panel). n=9-10 mice. Two-tailed t-test was used unless otherwise stated. All data is represented as mean±SEM.

FIG. 2. Histone H4 N-terminus induces stronger negative Gaussian curvature in cholesterol-rich membranes. FIG. 2(a) Rendering of H4n FIG. 2(b) Model of NGC generation by H4n FIG. 2(c) Representation of the Pn3m Q_{II} phase FIG. 2(d) SAXS spectra of H4n incubated with cholesterol-rich (PS/PE/CH 20/70/10) and cholesterol-poor (PS/PE 20/80) membranes at a P/L molar ratio of 1/40. H4n induces NGC in the form of a Pn3m Q_{II} phase. Cholesterol enhances the membrane activity of H4n. Observed reflections for the cubic (green), hexagonal (blue), and lamellar (red) phases have been assigned on the spectra.

FIG. 3. Apolipoprotein A-I mimic apoMP₁ inhibits the pro-lytic membrane activity of histone H4 N-terminus and unrelated peptides. FIG. 3(a) SAXS spectra of PS/PE/CH 20/70/10 SUVs incubated with H4n and apoMP₁. The P/L molar ratio of H4n was held constant at 1/40, while the P/L molar ratio of apoMP₁ was varied. H4n alone induced L_{α}, H_{II}, and Pn3m Q_{II} phases. Pn3m Q_{II} phases were suppressed at high concentrations of apoMP₁. FIG. 3(b) SAXS spectra of PS/PE 20/80 SUVs co-treated with the AMP PG-1 (NGC-generating peptide) and apoMP₁. The P/L molar ratio of PG-1 was held constant at 1/40, while the P/L molar ratio of apoMP₁ was varied. PG-1 alone induced L_{α}, H_{II}, Pn3m Q_{II}, and Im3m Q_{II} phases. Pn3m and Im3m Q_{II} phases were suppressed at high P/L molar ratios of apoMP₁, leaving behind a zero-curvature L_{α} phase. FIG. 3(c) SAXS spectra of PS/PE 20/80 SUVs co-treated with the CPP HIV-TAT (NGC-generating peptide) and apoMP₁. The P/L molar ratio of HIV-TAT was held constant at 1/40, while the P/L molar ratio of apoMP₁ was varied. HIV-TAT alone induced L_{α} and Pn3m Q_{II} phases. Pn3m Q_{II} phases were suppressed at high P/L molar ratios of apoMP₁, leaving behind L_{α} and H_{II} phases. (d) ApoMP₁ can transform an H_{II} phase with strong negative mean curvature into an L_{α} phase with zero mean curvature. FIG. 3(a-d) Observed reflections for the Pn3m cubic (green), Im3m cubic (yellow), hexagonal (blue), and lamellar (red) phases have been assigned on the curves. (e) Schematic portrayal of the suppression of H4n-induced NGC generation and pore formation by apoMP₁.

FIG. 4. Positive Gaussian curvature induced by peptides can restrain pores formed by negative Gaussian curvature-inducing peptides: FIG. 4(a) Barrier height of pore formation for mixtures of K>0 (orange) and K<0 (green)-inducing peptides or proteins in theoretical model (Supporting Information). Barrier without proteins E₀ given by E₀/k_{B}T=πΓ₀²/σ₀, with contours 10 k_{B}T apart. FIG. 4(b) Protein-prescribed curvatures mapped into corresponding equilibrium α_{Pn3m}, which grow with increasing molar ratio of K>0 to K<0-generating proteins, consistent with H4n/apoMP₁ experimental trends.

FIG. 5. ApoMP₁ blocks histone H4-mediated cytotoxicity and prevents organ tissue damage after LPS-induced endotoxemia. FIG. 5(a-d) Histone H4-induced cell death quantified by PI uptake in murine endothelial cells FIG. 5(a), smooth muscle cells FIG. 5(b), macrophages FIG. 5(c), and hepatocytes FIG. 5(d) treated with indicated doses of apoMP₁. n=13-31 fields. Results obtained from three independent experiments. One-way ANOVA with Dunnett's correction. *p<0.05; ***p<0.001; ****p<0.0001. FIG. 5(e, f) C57BL6/J mice were challenged with 10 mg/kg of LPS (E. coli O111:B4) for 24 hours and treated with apoMP₁ (250 μg/intraperitoneal) or vehicle (saline). Representative immunofluorescence micrographs showing TUNEL⁺ cells and quantification of percentage of TUNEL⁺ cells in liver FIG. 5(e) and lung FIG. 5(f). Scale bar: 50 μm. n=5 mice/group. Two-tailed t-test. All data is represented as mean±SEM.

FIG. 6. SAXS spectra for SUVs and buffer control samples. The SAXS profile of PS/PE 20/80 SUVs shows a form factor that is consistent with unilamellar vesicles and the SAXS spectrum of PS/PE/CH 20/70/10 SUVs indicates the existence of an H_{II} phase. Scattering from the buffer control sample shows background signal.

FIG. 7. Anti-pore peptide APP-2498 (also termed “AP1”) inhibits the pro-lytic membrane activity of histone H4 N-terminus and unrelated peptides. FIG. 7(a) SAXS spectra of PS/PE/CH 20/70/10 SUVs incubated with H4n and APP-2498. The P/L molar ratio of H4n was held constant at 1/40, while the P/L molar ratio of APP-2498 was varied. H4n alone induced L_{α}, H_{II}, and Pn3m Q_{II} phases. Pn3m Q_{II} phases were suppressed at high concentrations of APP-2498. FIG. 7(b) SAXS spectra of PS/PE 20/80 SUVs co-treated with the AMP melittin¹¹ (NGC-generating peptide) and APP-2498. The P/L molar ratio of melittin was held constant at 1/25, while the P/L molar ratio of APP-2498 was varied. Melittin alone induced Im3m Q_{II} phases. Im3m Q_{II} phases were suppressed at high P/L molar ratios of APP-2498, while a zero-curvature L_{α} phase gradually emerged. (c) SAXS spectra of PS/PE 20/80 SUVs co-treated with the CPP HIV-TAT (NGC-generating peptide) and APP-2498. The P/L molar ratio of HIV-TAT was held constant at 1/40, while the P/L molar ratio of APP-2498 was varied. HIV-TAT alone induced L_{α} and Pn3m Q_{II} phases. Pn3m Q_{II} phases were suppressed at high P/L molar ratios of APP-2498, leaving behind only L_{α} phases with no curvature. FIG. 7(a-c) Observed reflections for the Pn3m cubic (green), Im3m cubic (yellow), hexagonal (blue), and lamellar (red) phases have been assigned on the curves.

FIG. 8. Peptide-membrane interaction. FIG. 8(a, b) The interaction between a membrane with NGC and an anisotropic peptide depends on the relative angle between the membrane principal curvatures (black lines) and the peptide orientation. This difference is captured by Eq. (2). FIG. 8(c) On the other hand, an anisotropic peptide on a locally isotropic membrane, such as a sphere, can rotate without changing the energy cost. This additional rotational entropy competes with the energy cost of mismatched peptide and membrane curvature.

FIG. 9. Schematic of a model membrane pore. Peptides with NGC (represented by red, bent cylinders) favor pore formation by better matching the NGC of the inner rim of the pore while peptides with PGC (green spheres) do not. These two curvature types compete to raise or lower the free energy barrier for pore formation. The pore is modeled by the inner surface of a torus of large radius r and small radius 1, where t is the monolayer thickness.

FIG. 10 shows a helical wheel plot diagram that depicts the projection of the amino acid residue positions onto a plane perpendicular to the axis of the helix. Because an α-helix consists of 3.6 residues per turn, with a 100° angle between two consecutive residues, a periodicity exists for every 18 residues (5 turns). When depicted in a helical wheel plot, the peptide sequence features three distinct faces described as: nonpolar, polar, and positively charged. Each distinct face can be described by the angle subtended by its corresponding sector of residues, (e.g., the angle subtended by the nonpolar face is indicated by θ and its span is delineated by the dotted lines).

FIGS. 11A-11H provide graphed data from in vitro studies showing that the administration of peptides of the invention (AP1, AP2, AP4, AP7, AP8, AP9, AP10, AP11) results in the inhibition of Sindbis virus (SINV) viral replication with minimal cytotoxicity. Half-maximal effective concentrations (EC50) range from 0.98-26.23 μM. FIG. 11A shows data obtained using the AP1 peptide. FIG. 11B shows data obtained using the AP2 peptide. FIG. 11C shows data obtained using the AP4 peptide. FIG. 11D shows data obtained using the AP7 peptide. FIG. 11E shows data obtained using the AP8 peptide. FIG. 11F shows data obtained using the AP9 peptide. FIG. 11G shows data obtained using the AP10 peptide. FIG. 11H shows data obtained using the AP1 peptide.

FIGS. 12A-12B provide graphed data from in vitro studies of peptides of the invention peptides combined with HIV-TAT in order to demonstrate their ability to inhibit membrane remodeling. Each sample contains a mixture of liposomes, physiological salts, HIV-TAT (P/L=1/20), and an invention peptide at a specified P/L ratio (1/100, 1/50, or 1/25). All 7 invention peptides show dose-dependent inhibition of HIV-TAT-induced negative Gaussian curvature (NGC). FIG. 12A shows studies with AP1 (first panel), AP2 (second panel) and AP4 (third panel). FIG. 12B shows studies with AP7 (first panel), AP8 (second panel), AP9 (third panel) and AP10 (fourth panel).

FIGS. 13A-13B provide graphed data from studies of peptides of the invention peptides showing their ability to attenuate viral burden and lung injury induced by influenza infection. In these in vivo studies using a murine model of influenza, we show that the administration of peptides of the invention (AP1 and AP4) results in lower levels of M gene expression (FIG. 13A) as well as lower levels of BAL IgM (*, p<0.05) (FIG. 13B).

FIG. 14 provides graphed data from in vitro studies of peptides of the invention peptides showing their ability to protect against cytotoxicity induced by pro-inflammatory protein histone H4. These in vitro studies show that embodiments of the invention (AP1) can be used to increased level of cell viability (as determined by MTT assay on murine macrophages). This data provides evidence that embodiments of the invention can protect against cell death and associated inflammatory consequences caused by histone H4.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art.

As discussed below, the invention disclosed herein includes peptides that can exert inhibiting or counteracting membrane curvature effects that can inhibit the generation of negative Gaussian curvature, and thereby, inhibit membrane restructuring. The invention disclosed herein also includes methods of making and using such peptides.

Peptides having an ability to induce positive Gaussian curvature (and/or an ability to inhibit negative Gaussian curvature) can be applied to cell membranes (e.g., mammalian cell membranes) as therapeutic agents in order to address a number of significant healthcare needs. For example, by inhibiting membrane fusion between sperm and egg cells, such peptides can prevent fertilization and be used as a locally delivered non-hormonal contraceptive. In addition, for a virus to initiate infection, the virus must first gain access into the interior of a target host cell. Viral entry can occur either at the cell surface or from an internal compartment following endocytosis by the cell (64-66). For both enveloped and non-enveloped viruses, a key step in the entry process involves membrane remodeling. Enveloped viruses enter cells via membrane fusion, in which the viral membrane fuses with the host membrane, resulting in transfer of the core viral particle into the cytosol (67). In contrast, non-enveloped viruses form membrane pores to gain entry into the cell (68, 69). Therefore, by inhibiting the membrane restructuring processes that are required for a virus to enter a cell, the peptides of the invention can be used as a prophylactic antiviral against infections caused by enveloped and non-enveloped viruses.

Within a host cell, a virus hijacks the cell machinery to replicate its genetic information and assemble and release progeny virions to infect other cells and spread the viral infection. Shedding of virus particles can occur by budding through the cell plasma membrane, budding through an intracellular membrane and exiting the cell by exocytosis, and by lysing the host cell (70, 71), which are processes that remodel membranes by membrane fission, membrane fusion, and membrane pore formation. Thus, by inhibiting these membrane processes, such peptides can be used as an antiviral therapeutic (against enveloped and non-enveloped viral infections) to mitigate the spread of infection within a host and viral transmission between individuals.

In addition, non-programmed cell death results in uncontrolled release of intracellular contents, which stimulate an inflammatory response (72). The ability of the peptides disclosed herein to inhibit lytic membrane restructuring can reduce the severity and duration of inflammation induced by viral infections and other biological triggers, including, but not limited to, those disclosed herein. While many viruses intrinsically cause cell lysis and tissue damage, viral infections also evoke an antiviral immune response that leads to increased cellular destruction and inflammation (73, 74). Histones released from neutrophils have been recently shown to induce cell lysis and identified as a source of chronic inflammation (31).

In addition, membrane-bound secretory vesicles fuse with the plasma membrane of a cell to release their contents to the cell exterior. This process of occurs through exocytosis, which requires membrane fusion. By inhibiting membrane fusion, the peptides of the invention can reduce the secretion of neurotransmitters or hormones to treat neuropsychiatric or endocrine conditions. For example, the peptides of the invention may be useful as an anticonvulsant or antiepileptic drug by reducing the release of excitatory glutamate, which is elevated in epilepsy (75, 76).

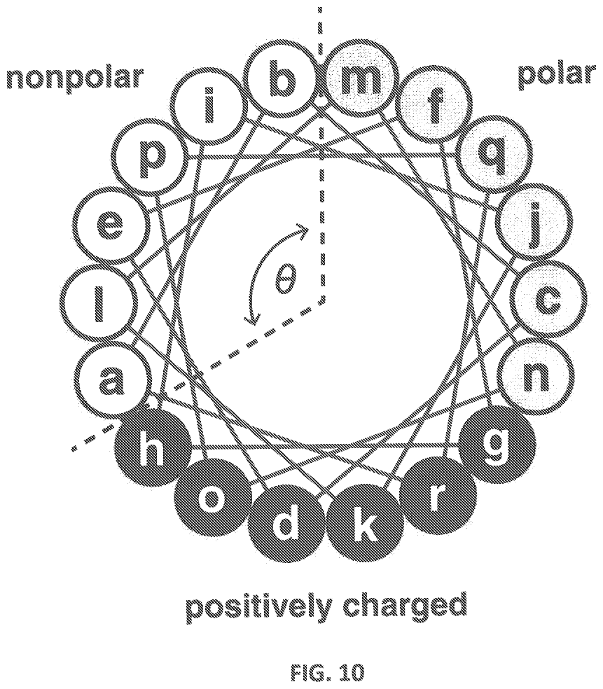

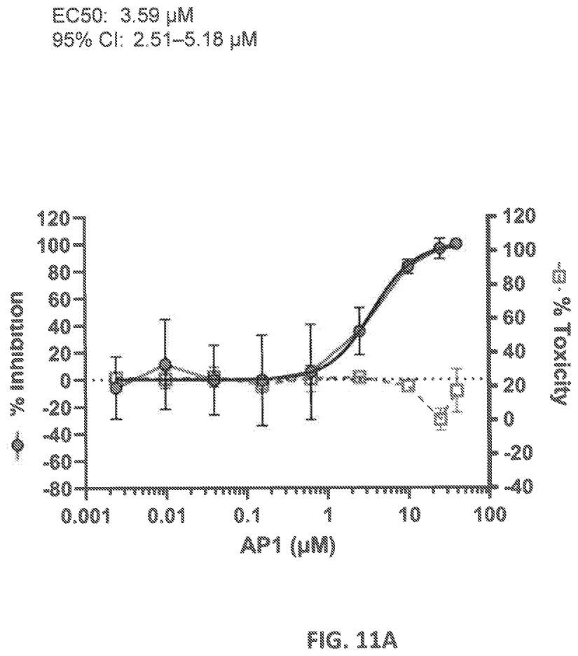

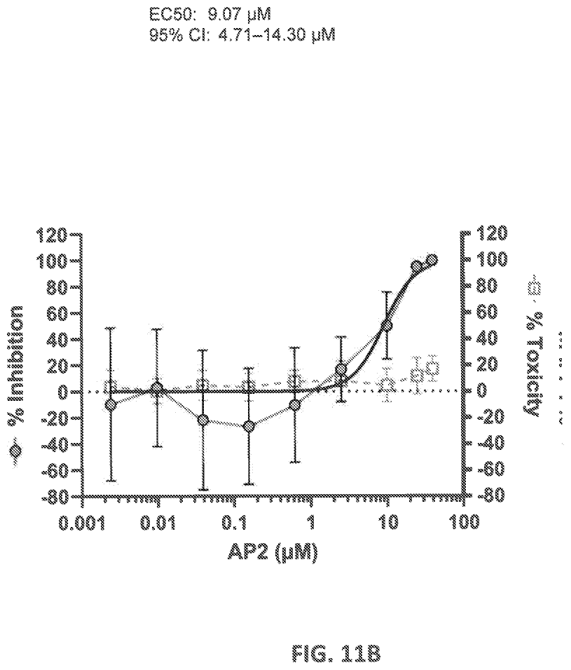

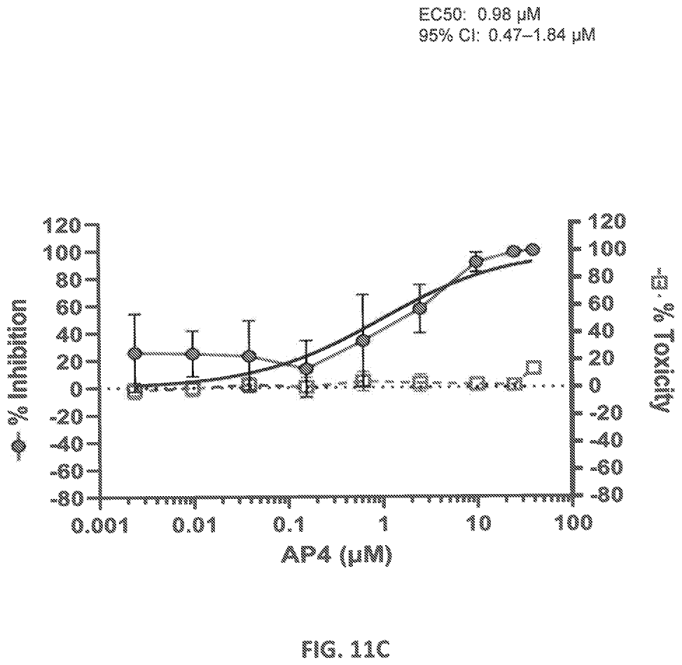

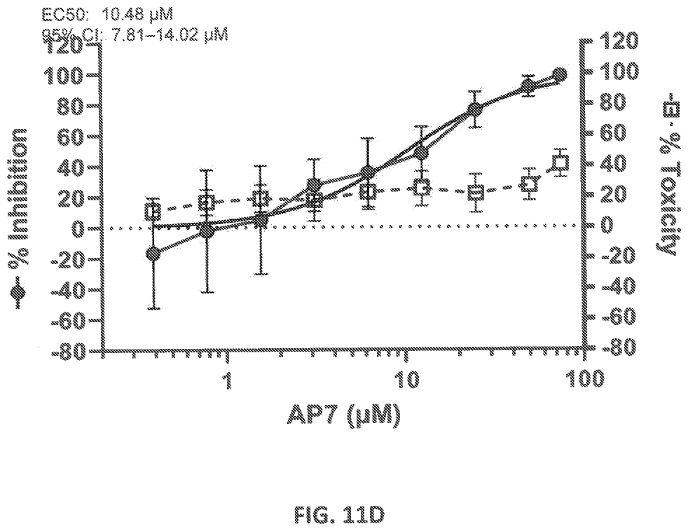

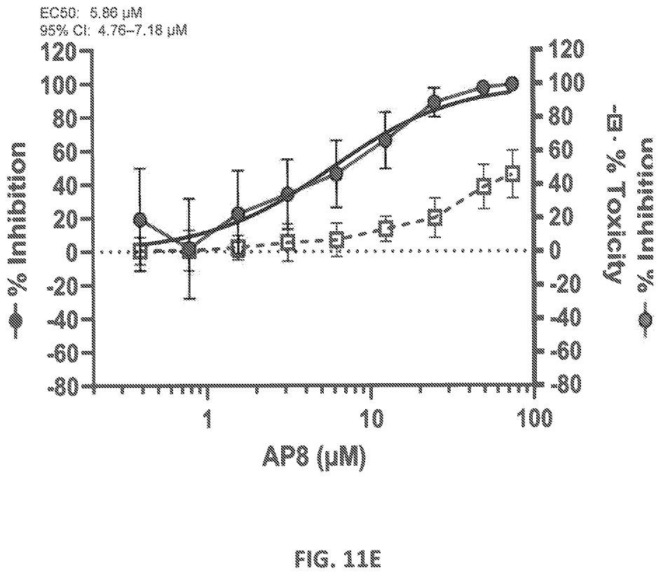

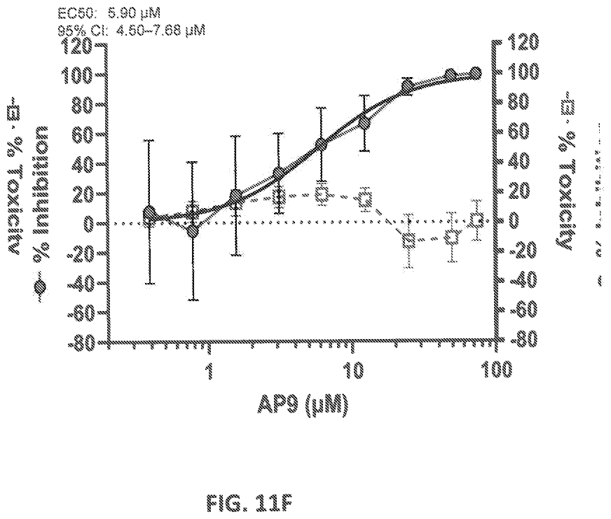

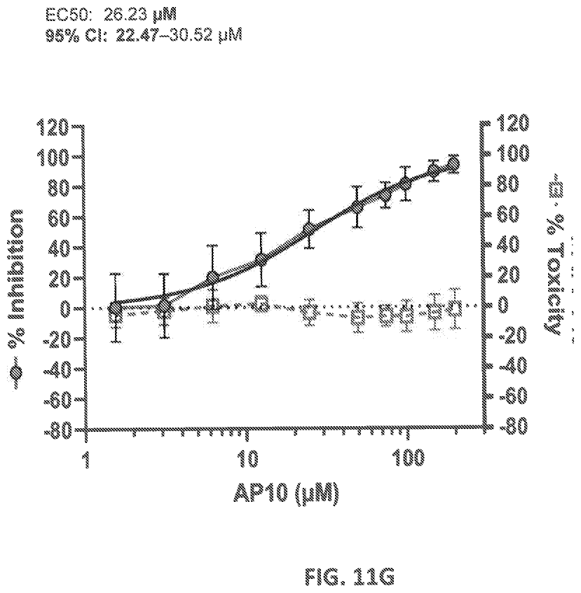

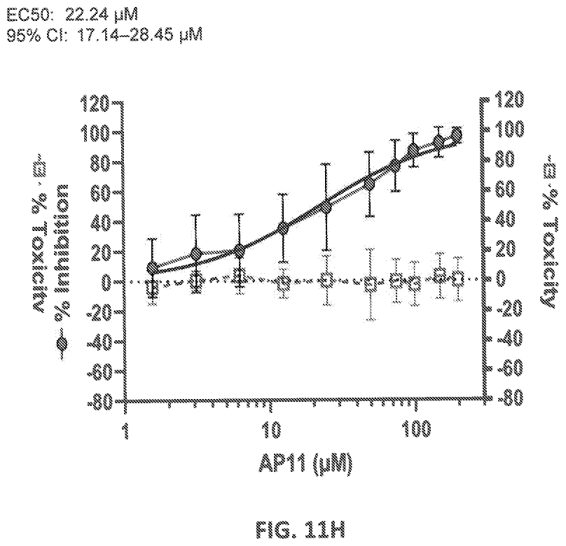

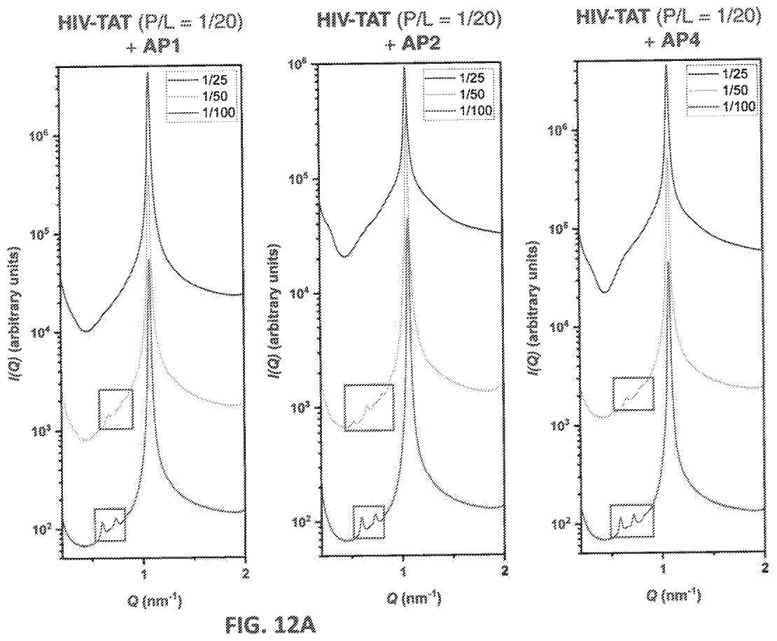

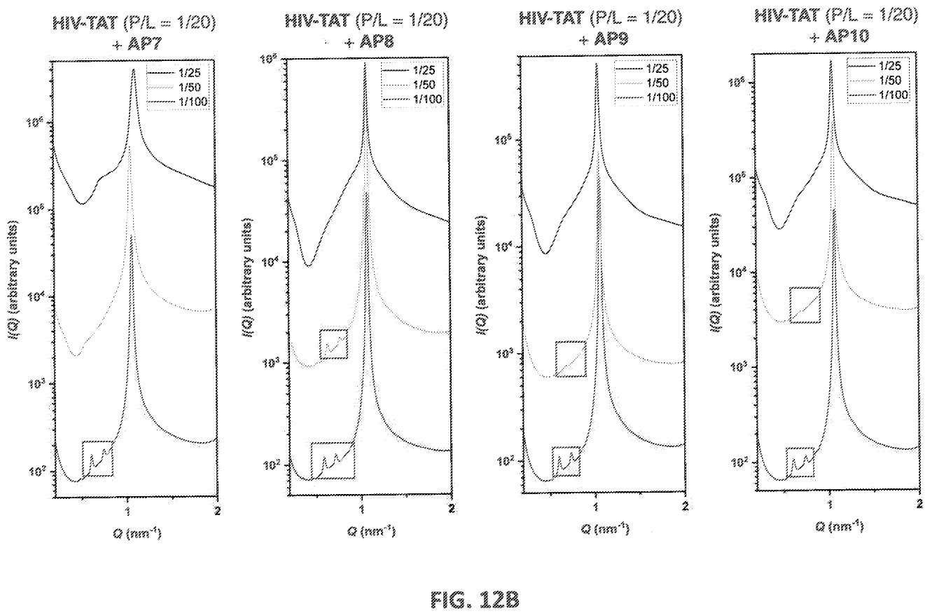

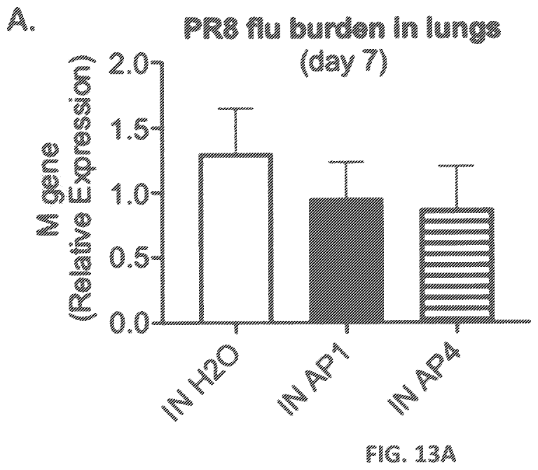

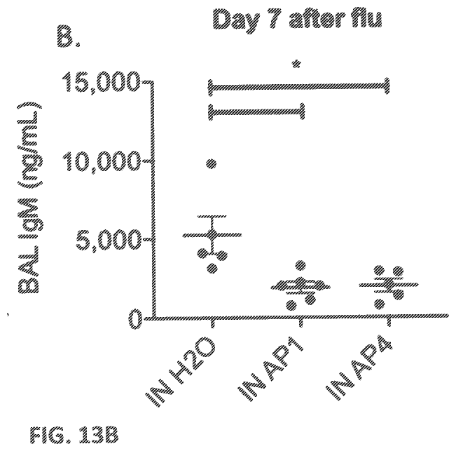

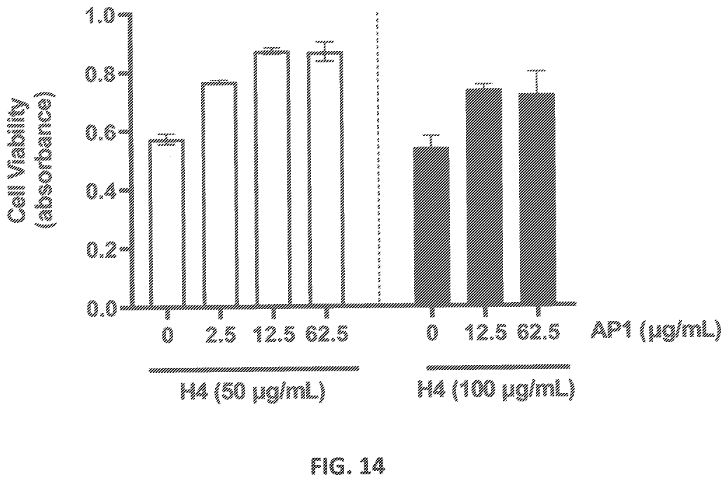

The invention disclosed herein has a number of embodiments. Embodiments of the invention include, for example, a process for making a peptide product from a plurality of amino acid residues that form a peptide designed to have an ability to induce positive Gaussian curvature in a mammalian cell contacted with a peptide of the invention and/or an ability to inhibit negative Gaussian curvature in a mammalian cell contacted with the peptide. Typically, the process comprises forming a peptide comprising a helix having an axis from a plurality of amino acids selected such that when visualized in a helical wheel plot diagram (see, e.g., FIG. 10) depicting projections of amino acid residue positions onto a plane that is perpendicular to the axis of the helix of the peptide, the peptide has a number of selected features. For example, in this process, the peptide sequence can be designed to comprise a plurality of amino acid residues selected to form three segments of amino acids in the peptide product, wherein the three segments of amino acids comprise: a nonpolar segment of amino acid residues, a polar segment of amino acid residues, and a segment of positively charged amino acid residues under physiological conditions. Typically in this process for making the peptide product: (1) the nonpolar segment comprises 6-11 continuous amino acid residues which together subtend a radial angle of 120-220° perpendicular to the axis of the helix; and the nonpolar segment comprises at least 4 amino acid residues selected from A, C, F, G, I, L, M, P, V, W and Y; (2) the polar segment comprises 4-13 continuous amino acid residues which together subtend a radial angle of 80-260° perpendicular to the axis of the helix; and the polar segment comprises at least 4 amino acid residues selected from A, C, D, E, G, H, K, N, P, Q, R, S, T and Y; and (3) the positively charged segment comprises 1-6 continuous amino acid residues which together subtend a radial angle of 20-120° perpendicular to the axis of the helix; and the positively charged segment comprises at least 1 amino acid residue selected from H, K and R. In this context, artisans understand that “continuous amino acid residues which together subtend a radial angle of 120-220” means that this segment of amino acid residues in a peptide spans at least 120° on a helical wheel plot diagram (or ⅓ of the 360° total in the helical wheel plot diagram) and not more than 220° on a helical wheel plot diagram. Upon the selection of the amino acids following these parameters, the peptide product is formed from the plurality of selected amino acids residues (e.g., using a conventional methodology) so that the peptide product is made. In certain embodiments of the invention, the peptide is size selected so as to comprise not more than 50 amino acids, or not more than 25 amino acids or not more than 20 amino acids.

In certain embodiments of the invention, the peptide product is selected to comprises least one amino residue selected from: G, N, Q and S; and/or the peptide product is formed so that it does not comprise more than 5 aromatic nonpolar amino acid residues; and/or the peptide product is designed to exhibit a net charge between −3 and +3 at physiological pH. In certain embodiments of the invention, the peptide is then coupled to a plurality of additional amino acids, such as one found in a polypeptide sequence expressed by a mammalian genome. In certain embodiments of the invention, a peptide product does not have the sequence: D-W-F-K-A-F-Y-D-K-V-A-E-K—F-K-E-A-F (SEQ ID NO: 2). In certain embodiments of the invention, a peptide product comprises the sequence: G-D-A-V-R-E-W-F-E-K-A-W-Q-R-V-R-E-F-F (“APP” SEQ ID NO: 1), FFERVRQWAKEFWERVADG (“AP2” SEQ ID NO: 7), GDAVREVIEKAVQRVREIV (“AP3” SEQ ID NO: 8), GDAVKEWFEKAWQKVKEFF (“AP4” SEQ ID NO: 9), GERAKEWVEAFWEKAREYF (“AP5” SEQ ID NO: 10), GERVKEFFEAFFEKAREYW (“AP6” SEQ ID NO: 11), GEKAKEWVQAFWQKAKEYF (“AP7” SEQ ID NO: 12), GEKVKEFFQAFFQKAKEYW (“AP8” SEQ ID NO: 13), GDAVKEWFEKAWQKVKEFL (“AP9” SEQ ID NO: 14), GEQLKQKFQEFWDKLKEWY (“AP10” SEQ ID NO: 15), GEKLKQKAQEFFDAVKEWF (“AP11” SEQ ID NO: 16), GKEKAEEFFQALKEWFDKFKN (“AP12” SEQ ID NO: 17) or GEQFKQAFQEWWDKLKEKY (“AP13” SEQ ID NO: 18). Certain embodiments of the invention further comprise combining the peptide product with a pharmaceutically acceptable carrier.

Embodiments of the invention also include peptide products and associated peptide compositions made by a process disclosed herein. In certain embodiments of the invention, the peptide induces positive Gaussian curvature in a mammalian cell contacted with the peptide product and/or counteracts negative Gaussian curvature in a mammalian cell contacted with the peptide product. Embodiments of the invention also include compositions of matter comprising a polynucleotide encoding a peptide made by the process disclosed herein (e.g. G-D-A-V-R-E-W-F-E-K-A-W-Q-R-V-R-E-F-F (“AP1” SEQ ID NO: 1), FFERVRQWAKEFWERVADG (“AP2” SEQ ID NO: 7), GDAVREVIEKAVQRVREIV (“AP3” SEQ ID NO: 8), GDAVKEWFEKAWQKVKEFF (“AP4” SEQ ID NO: 9), GERAKEWVEAFWEKAREYF (“AP5” SEQ ID NO: 10), GERVKEFFEAFFEKAREYW (“AP6” SEQ ID NO: 11), GEKAKEWVQAFWQKAKEYF (“AP7” SEQ ID NO: 12), GEKVKEFFQAFFQKAKEYW (“AP8” SEQ ID NO: 13), GDAVKEWFEKAWQKVKEFL (“AP9” SEQ ID NO: 14), GEQLKQKFQEFWDKLKEWY (“AP10” SEQ ID NO: 15), GEKLKQKAQEFFDAVKEWF (“AP11” SEQ ID NO: 16), GKEKAEEFFQALKEWFDKFKN (“AP12” SEQ ID NO: 17) or GEQFKQAFQEWWDKLKEKY (“AP13” SEQ ID NO: 18). In typical embodiments, the polynucleotide encoding the peptide is fused in frame to a polynucleotide sequence encoding a plurality of amino acids. Optionally the polynucleotide encoding the peptide is disposed within a vector comprising sequences for expressing the peptide in a bacterial, yeast or mammalian cell.

Other embodiments of the invention include methods of inducing positive Gaussian curvature in a mammalian cell and/or counteracting negative Gaussian curvature in a mammalian cell, the method comprising contacting the mammalian cell with a composition comprising a peptide made by a process disclosed herein, wherein concentrations of the peptide in the composition are selected to be sufficient to induce positive Gaussian curvature in the mammalian cell and/or counteract negative Gaussian curvature in the mammalian cell when the mammalian cell is contacted with the composition. In some embodiments of the invention, the method is adapted to prevent fertilization by inhibiting membrane fusion between sperm and egg cells. In some embodiments of the invention, the method is adapted to inhibit viral replication processes requiring negative Gaussian membrane curvature. In some embodiments of the invention, the method is adapted to mitigate virus-associated inflammation. In some embodiments of the invention, the method is adapted to mitigate inflammation resulting from lytic cell death or tissue damage by inhibiting permeabilization of cell membranes. In certain embodiments of the invention, the method is adapted to interfere with and/or inhibit membrane fusion required for secretory processes, including, but not limited to, neurotransmitter release, hormone secretion, and enzyme release; and/or to stabilize natural or artificial lipid membranes so as to inhibit said membrane permeabilization.

As is known in the art, a helical wheel is a type of plot or visual representation used to illustrate the properties of α-helices in proteins. In this art accepted plot or visual representation, sequences of amino acids that make up a helical region of the protein's secondary structure are plotted in a rotating manner where the angle of rotation between consecutive amino acids is 100°, so that the final representation looks down the helical axis. The plot typically reveals whether hydrophobic amino acids are concentrated on one side of the helix, usually with polar or hydrophilic amino acids on the other. This arrangement is common in α-helices within globular proteins, where one face of the helix is oriented toward the hydrophobic core and one face is oriented toward the solvent-exposed surface. Specific patterns characteristic of protein folds and protein docking motifs are also revealed, as in the identification of leucine zipper dimerization regions and coiled coils. This projection diagram is often called and “Edmondson wheel” after its inventor. (see, also, Schiffer M, Edmundson A B (1967). “Use of helical wheels to represent the structures of proteins and to identify segments with helical potential”. Biophysical Journal. 7 (2): 121-35. Bibcode:1967BpJ.7.121S. doi:10.1016/S0006-3495(67)86579-2. PMC 1368002. PMID 6048867; Wadhwa, R; Subramanian, V; Stevens-Truss, R (2018). “Visualizing alpha-helical peptides in R with helixvis”. Journal of Open-Source Software. 3 (31): 1008. Bibcode:2018JOSS . . . 3.1008W. doi:10.21105/joss.01008; and Gautier, R; Douguet, D; Antonny, B; Drin, G (2008). “HELIQUEST: a web server to screen sequences with specific alpha-helical properties”. Bioinformatics. 24 (18): 2101-2102. doi:10.1093/bioinformatics/btn392. PMID 18662927).

Typically, peptides of the invention contain an amino acid sequence that allows for the peptide to adopt an amphipathic α-helical structure, in which the physicochemical properties of the amino acid residues form distinct faces oriented along the axis of the helix. When envisioned as an amphipathic α-helix in a helical wheel plot, the peptides made by the methods disclosed herein typically feature three faces that are described as nonpolar, polar, and positively charged.

In certain embodiments of the invention, a helical wheel plot is used to design peptides by depicting the projection of the amino acid residue positions onto a plane perpendicular to the axis of the helix. Because an α-helix consists of 3.6 residues per turn, with a 1000 angle between two consecutive residues, a periodicity exists for every 18 residues (5 turns). When depicted in a helical wheel plot (e.g., as shown in FIG. 10), the peptide sequences of the invention feature three distinct faces that are described as: nonpolar, polar, and positively charged. Each distinct face can be described by the angle subtended by its corresponding sector of residues, (e.g., in FIG. 10, the angle subtended by the nonpolar face is indicated by 0 and its span is delineated by the dotted lines). A description of each of the three faces of peptides formed by the methods disclosed herein is as follows:
(1) The nonpolar face consists of 6-11 adjacent residues on the helical wheel plot, which together subtend a radial angle (perpendicular to the axis of the helix) of 120-220°. The nonpolar face contains 4 or more residues that are classified as nonpolar amino acids, which are defined here as being any of the following: A, C, F, G, I, L, M, P, V, W, Y
(2) The polar face consists of 4-13 adjacent residues on the helical wheel plot, which together subtend a radial angle (perpendicular to the axis of the helix) of 80-260°. The polar face contains 4 or more residues that are classified as polar amino acids, which are defined here as being any of the following: A, C, D, E, G, H, K, N, P, Q, R, S, T, Y
(3) The positively charged face consists of 1-6 adjacent residues on the helical wheel plot, which together subtend a radial angle (perpendicular to the axis of the helix) of 20-120°. The positively charged face contains 1 or more residues that are classified as positively charged amino acids, which are defined here as being any of the following: H, K, R.

In some embodiments of the invention, the peptide must contain at least one of the following residues: G, N, Q, S. In some embodiments of the invention, the peptide does not contain more than 5 aromatic nonpolar residues (F, W, Y). In certain embodiments of the invention, the peptide exhibits a net charge between −3 and +3 at physiological pH.

In certain embodiments of the invention, concentrations of the peptide in the composition are selected to be sufficient to induce positive Gaussian curvature in a mammalian cell contacted with the composition; and/or concentrations of the peptide in the composition are selected to be sufficient to counteract negative Gaussian curvature in a mammalian cell contacted with the composition. In illustrative embodiments of the invention, a peptide is selected or designed such that a peptide:lipid molar ratio of 1/10 or lower can inhibit the generation of NGC magnitudes |<K>| that are 1×10⁻³ nm⁻² or greater in lipid membranes composed of 20/80 1,2-dioleoyl-sn-glycero-3-phospho-L-serine (CAS number: 90693-88-2)/1,2-dioleoyl-sn-glycero-3-phosphoethanolamine (CAS number: 4004-05-1).

In FIG. 10, an example peptide sequence “abcdefghijklmnopqr” is shown in a helical wheel plot. In this particular example, the three faces have an equal number of residues. The nonpolar face (white residues in FIG. 10) consists of the adjacent residues: a, 1, e, p, i, b. The polar face (grey residues in FIG. 10) consists of the adjacent residues: m, f, q, j, c, n. The positively charged face (black residues in FIG. 10) consists of the adjacent residues: g, r, k, d, o, h.

Here the terms “peptide” and “protein” are interchangeable to refer to a polymer of amino acid residues. These terms apply to both amino acid polymers in which one or more amino acid residues is a natural amino acid or a chemical analog of a natural amino acid, which includes, but is not limited to, both L- and D-amino acid enantiomers. The peptides in this invention are chemically synthesized or recombinantly expressed. The amino terminus of the peptide can be protected by an acetyl group or another protecting group. The carboxyl terminus of the peptide can be protected by an amide group or another protecting group. Cleavable linkers or bonds can be incorporated between amino acid residues to allow for controlled degradation.

### Illustrative Uses for Embodiments of the Invention

The peptides disclosed herein can be formulated into a wide variety of specific formulations, including, but not limited to, eye drops, nasal spray, aerosol inhaler or nebulizer, injection, time-release implant, suppository, or oral form (e.g., as a capsule, tablet, syrup, lozenge), and can be administered systemically or locally. For example, certain embodiments of the compositions of the invention include a pharmaceutical excipient such as one selected from the group consisting of a preservative, a tonicity adjusting agent, a detergent, a viscosity adjusting agent, a sugar and a pH adjusting agent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

Embodiments of the invention disclosed herein can be used to addresses multiple unmet healthcare needs, for example as non-hormonal contraceptives. In the United States each year, nearly 45% (or 2.8 million) of all pregnancies are unintended (1). Over 99% of sexually active reproductive-aged women in the United States have used at least one form of contraception (2). The 2015-2017 National Survey of Family Growth found that 64.9% of 72.2 million women aged 15-49 in the United States were currently using contraception (3). Among these women, the most common contraceptives used were non-permanent hormonal methods (3, 4). Hormonal contraceptives are currently the most effective reversible contraceptive method, with rates of unintended pregnancies<10% for typical use, in comparison to non-hormonal methods, which have rates that range from 12-28% (5, 6). However, women who use hormonal contraceptive methods frequently experience unwanted side effects, such as nausea, weight gain, mood changes, and headaches (7), in addition to having increased risks of breast cancer (8, 9), cardiovascular disease (10), blood clots (11), and depression (12). The development of more effective non-hormonal contraceptive options can potentially offer women protection against unintended pregnancies at reduced risk of adverse health effects.

Embodiments of the invention disclosed herein can be used as broad-spectrum antiviral agents. Viruses that cause emerging infectious diseases pose an enormous threat to global health. Conventional virus specific vaccines and treatments are becoming an inefficient approach against the increasing diversity of viral pathogens, especially those that pose epidemic and pandemic risks. Viruses are classified as either enveloped viruses, which possess a lipid membrane (envelope), or non-enveloped viruses, which lack a membrane. The vast majority of highly pathogenic viral infections with severe illness and high mortality rates, including those caused by Ebola virus (EBOV), influenza viruses, and coronaviruses (such as Middle East respiratory coronavirus (MERS CoV), severe acute respiratory syndrome coronavirus (SARS-CoV), and severe acute respiratory syndrome coronavirus 2 (SARS-CoV-2)), are from enveloped viruses (13). Compared with enveloped viruses, non-enveloped viruses (such as coxsackieviruses, noroviruses, rhinoviruses, Hepatovirus A (HAV), and human papillomavirus (HPV)), are generally associated with lower mortality rates, but are more likely to exhibit human-to-human transmission (14, 15) and have a greater chance of environmental spread due to higher surface survival and lower susceptibility to chemical disinfectants (16, 17). Severity and transmissibility are two major factors that contribute to the threat from infectious viral diseases. However, the absence of effective broad-spectrum antiviral agents plays an important role. Current antivirals offer limited spectrum of activity and capacity for prompt control of epidemic viral infections, which underscores the urgent and imperative need for broad-spectrum antiviral agents that can act against multiple viruses (13, 18-21). In spite of the diversity among viruses, there exist fundamental processes and features that are common to enveloped and/or non-enveloped viruses. Targeting these conserved elements opens new avenues for the design of broad-spectrum antivirals.

Embodiments of the invention disclosed herein can be used in anti-inflammatory therapies against newly identified mechanisms of inflammation. Excessive, uncontrolled inflammatory responses can increase susceptibility to secondary infections and promote disease pathogenesis. Currently, corticosteroids and non-steroidal anti-inflammatory drugs (NSAIDs) are used to reduce inflammation. While effective, both have mild to serious side effects (22-26). In addition, corticosteroids act as immunosuppressants, which carry a higher risk of developing infections and reduce the effectiveness of vaccines (27-30). Thus, there is need for anti-inflammatory therapeutics that can be effective against recently identified mechanisms of inflammation (31) and that ideally present fewer of side effects.

Embodiments of the invention disclosed herein can be used as therapeutic agents that modulate secretion of neurotransmitters and hormones. Imbalances of neurotransmitters have been associated with a variety of disease conditions and mental disorders. For example, elevated concentrations of the excitatory neurotransmitter glutamate have been implicated in a number of central nervous system disorders, including Alzheimer's disease, Parkinson's disease, Huntington's disease, amyotrophic lateral sclerosis, epilepsy, major depressive disorder, and anxiety disorders (32-38).

Neurodegenerative diseases are debilitating and incurable conditions that result from the gradual and progressive loss of neurons in the central nervous system, leading to motor and cognitive impairments. Largely due to the increased life expectancy, neurodegenerative disorders are becoming more prevalent, as the risk increases dramatically with age. Alzheimer's and Parkinson's diseases are the most common neurodegenerative diseases, affecting over 43 million (39) and 6 million (40) people worldwide, respectively. Recent work has indicated that toxic overactivation of glutamate receptors contributes to the synaptic dysfunction and neuronal loss associated with neurodegenerative diseases such as Alzheimer's disease, Parkinson's disease, Huntington's disease, and amyotrophic lateral sclerosis (32, 33, 41-43). This finding has led to current therapeutic strategies, in which the effects from high glutamate levels are blocked via receptor antagonists (such as MK801 (dizocilpine), phencyclidine, ketamine, and memantine) and inhibitors of both glutamate release and postsynaptic glutamate receptor signaling (such as riluzole) (44-46). However, while effective, many of these also cause severe side effects. In fact, clinical trials of riluzole were completed for the treatment of Parkinson's (47) and Huntington's (48) diseases but were met with negative results. Because the majority of current treatments for neurodegenerative diseases aim to alleviate symptoms, new therapies that can slow disease progression are a major unmet need to combat the societal, emotional, and economic costs of these diseases as our population ages (49).

As one of the most common neurological conditions, epilepsy affects approximately 3 million people in the U.S. and 50 million people worldwide (50, 51). Epilepsy is characterized by spontaneous and recurrent seizures, in which increased levels of glutamate are believed to play a key role (35, 52). Anticonvulsants are the main treatment for epilepsy patients to prevent seizures. While these drugs are effective for many patients, challenges remain, including drug-resistant epilepsy and drug interactions (53, 54). Major depressive disorder, also known more simply as depression, is a debilitating illness that is estimated to affect over 300 million people globally (37). Elevated levels of glutamate have been found in distinct regions of the brain in patients with major depressive disorders (36). Because existing therapies are insufficient for many individuals, there is an urgent need to improve the management and treatment of major depressive disorder with new agents that are effective, rapid acting, and better tolerated (36).

Anxiety disorders are the most prevalent of psychiatric disorders, affecting up to 18.1% of individuals in the United States alone (55). While current treatments, which primarily target GABA and serotonergic neurotransmission, can be effective, they often have adverse side effects (56). A growing body of work has indicated that stress is a key contributing factor in developing anxiety disorders, and that excitatory glutamatergic neurotransmission is enhanced by stress (57). Evidence has shown that drugs that alter glutamate transmission have potential to reduce anxiety symptoms (57, 58).

### REFERENCES LISTED ABOVE

(1) L. B. Finer, M. R. Zolna, Declines in Unintended Pregnancy in the United States, 2008-2011. New England Journal of Medicine 374:9, 843-852 (2016).
(2) K. Daniels, W. D. Mosher, J. Jones, “Contraceptive methods women have ever used: United States, 1982-2010,” National health statistics reports No. 62 (National Center for Health Statistics, Hyattsville, M D, 2013).
(3) K. Daniels, J. C. Abma, “Current contraceptive status among women aged 15-49: United States, 2015-2017,” NCHS Data Brief No. 327 (National Center for Health Statistics, Hyattsville, M D, 2018).
(4) M. L. Kavanaugh, J. Jerman, Contraceptive method use in the United States: trends and characteristics between 2008, 2012 and 2014. Contraception 97:1, 14-21 (2018).
(5) J. Trussell, Contraceptive failure in the United States. Contraception 83:5, 397-404 (2011).
(6) A. Sundaram et al., Contraceptive Failure in the United States: Estimates from the 2006-2010 National Survey of Family Growth. Perspectives on Sexual and Reproductive Health 49:1, 7-16 (2017).
(7) D. J. Hooper, Attitudes, Awareness, Compliance and Preferences among Hormonal Contraception Users. Clinical Drug Investigation 30:11, 749-763 (2010).
(8) E. E. Calle et al., Breast cancer and hormonal contraceptives: further results: Collaborative group on hormonal factors in breast cancer. Contraception 54:3,
Supplement 1, 1-106 (1996).
(9) L. S. Morch et al., Contemporary Hormonal Contraception and the Risk of Breast Cancer. New England Journal of Medicine 377:23, 2228-2239 (2017).
(10) C. L. Shufelt, C. N. Bairey Merz, Contraceptive Hormone Use and Cardiovascular Disease. Journal of the American College of Cardiology 53:3, 221-231 (2009).
(11) B. H. Stegeman et al., Different combined oral contraceptives and the risk of venous thrombosis: systematic review and network meta-analysis. BMJ: British Medical Journal 347, f5298 (2013).
(12) C. W. Skovlund, L. S. Morch, L. V. Kessing, T. Lange, Ø. Lidegaard, Association of Hormonal Contraception With Suicide Attempts and Suicides. American Journal of Psychiatry 175:4, 336-342 (2018).
(13) F. Vigant, N. C. Santos, B. Lee, Broad-spectrum antivirals against viral fusion. Nature Reviews Microbiology 13:7, 426-437 (2015).
(14) J. L. Geoghegan, A. M. Senior, F. Di Giallonardo, E. C. Holmes, Virological factors that increase the transmissibility of emerging human viruses. Proceedings of the National Academy of Sciences 113:15, 4170-4175 (2016).
(15) J. J. Bull, A. S. Lauring, Theory and Empiricism in Virulence Evolution. PLOS Pathogens 10:10, e1004387 (2014).
(16) S. A. Sattar, Hierarchy of Susceptibility of Viruses to Environmental Surface Disinfectants: A Predictor of Activity Against New and Emerging Viral Pathogens. Journal of AOAC INTERNATIONAL 90:6, 1655-1658 (2007).
(17) S. Firquet et al., Survival of Enveloped and Non-Enveloped Viruses on Inanimate Surfaces. Microbes and Environments 30:2, 140-144 (2015).
(18) J.-D. Zhu, W. Meng, X.-J. Wang, H.-C. R. Wang, Broad-spectrum antiviral agents. Frontiers in Microbiology 6:517, (2015).
(19) M. C. Wolf et al., A broad-spectrum antiviral targeting entry of enveloped viruses. Proceedings of the National Academy of Sciences 107.7, 3157-3162 (2010).
(20) J. F. W. Chan et al., Broad-spectrum antivirals for the emerging Middle East respiratory syndrome coronavirus. Journal of Infection 67:6, 606-616 (2013).
(21) M. J. Aman et al., Development of a broad-spectrum antiviral with activity against Ebola virus. Antiviral Research 83:3, 245-251 (2009).
(22) I. L. Meek, M. A. F. J. Van de Laar, H. E. Vonkeman, Non-Steroidal Anti-Inflammatory Drugs: An Overview of Cardiovascular Risks. Pharmaceuticals 3:7, 2146-2162 (2010).
(23) W. A. Ray et al., COX-2 selective non-steroidal anti-inflammatory drugs and risk of serious coronary heart disease. The Lancet 360:9339, 1071-1073 (2002).
(24) L. S. Coles, J. F. Fries, R. G. Kraines, S. H. Roth, From experiment to experience: Side effects of nonsteroidal anti-inflammatory drugs. The American Journal of Medicine 74:5, 820-828 (1983).
(25) T. Rhen, J. A. Cidlowski, Antiinflammatory Action of Glucocorticoids—New Mechanisms for Old Drugs. New England Journal of Medicine 353:16, 1711-1723 (2005).
(26) R. Dahl, Systemic side effects of inhaled corticosteroids in patients with asthma. Respiratory Medicine 100:8, 1307-1317 (2006).
(27) C. Andrejak et al., Chronic respiratory disease, inhaled corticosteroids and risk of non-tuberculous mycobacteriosis. Thorax 68:3, 256-262 (2013).
(28) F. N. Aberra et al., Corticosteroids and immunomodulators: postoperative infectious complication risk in inflammatory bowel disease patients. Gastroenterology 125:2, 320-327 (2003).
(29) C.-H. Lee et al., Use of inhaled corticosteroids and the risk of tuberculosis. Thorax 68:12, 1105-1113 (2013).
(30) T. T. Sytsma, L. K. Greenlund, L. S. Greenlund, Joint Corticosteroid Injection Associated With Increased Influenza Risk. Mayo Clinic Proceedings: Innovations, Quality & Outcomes 2:2, 194-198 (2018).
(31) C. Silvestre-Roig et al., Externalized histone H4 orchestrates chronic inflammation by inducing lytic cell death. Nature 569:7755, 236-240 (2019).
(32) D. W. Choi, Glutamate neurotoxicity and diseases of the nervous system. Neuron 1:8, 623-634 (1988).
(33) S. A. Lipton, P. A. Rosenberg, Excitatory Amino Acids as a Final Common Pathway for Neurologic Disorders. New England Journal of Medicine 330:9, 613-622 (1994).
(34) T. Miladinovic, M. G. Nashed, G. Singh, Overview of Glutamatergic Dysregulation in Central Pathologies. Biomolecules 5:4, 3112-3141 (2015).
(35) M. J. During, D. D. Spencer, Extracellular hippocampal glutamate and spontaneous seizure in the conscious human brain. The Lancet 341:8861, 1607-1610 (1993).
(36) D. C. Mathews, I. D. Henter, C. A. Zarate, Targeting the Glutamatergic System to Treat Major Depressive Disorder. Drugs 72:10, 1313-1333 (2012).
(37) E. Pitsillou el al., The cellular and molecular basis of major depressive disorder: towards a unified model for understanding clinical depression. Molecular Biology Reports 47:1, 753-770 (2020).
(38) P. Bittigau, C. Ikonomidou, Topical Review: Glutamate in Neurologic Diseases. Journal of Child Neurology 12:8, 471-485 (1997).
(39) E. Nichols et al., Global, regional, and national burden of Alzheimer's disease and other dementias, 1990-2016: a systematic analysis for the Global Burden of Disease Study 2016. The Lancet Neurology 18:1, 88-106 (2019).
(40) E. R. Dorsey et al., Global, regional, and national burden of Parkinson's disease, 1990-2016: a systematic analysis for the Global Burden of Disease Study 2016. The Lancet Neurology 17:11, 939-953 (2018).
(41) J. Liu, L. Chang, Y. Song, H. Li, Y. Wu, The Role of NMDA Receptors in Alzheimer's Disease. Frontiers in Neuroscience 13:43, (2019).
(42) M. P. Parsons, L. A. Raymond, Extrasynaptic NMDA Receptor Involvement in Central Nervous System Disorders. Neuron 82:2, 279-293 (2014).
(43) J. Lewerenz, P. Maher, Chronic Glutamate Toxicity in Neurodegenerative Diseases-What is the Evidence? Frontiers in Neuroscience 9:469, (2015).
(44) S. A. Lipton, Paradigm shift in neuroprotection by NMDA receptor blockade: Memantine and beyond. Nature Reviews Drug Discovery 5:2, 160-170 (2006).
(45) W. V. Graham, A. Bonito-Oliva, T. P. Sakmar, Update on Alzheimer's Disease Therapy and Prevention Strategies. Annual Review of Medicine 68:1, 413-430 (2017).
(46) X. Song et al., Mechanism of NMDA receptor channel block by MK-801 and memantine. Nature 556:7702, 515-519 (2018).
(47) G. Bensimon et al., Riluzole treatment, survival and diagnostic criteria in Parkinson plus disorders: The NNIPPS Study. Brain 132:1, 156-171 (2008).
(48) G. B. Landwehrmeyer et al., Riluzole in Huntington's disease: a 3-year, randomized controlled study. Annals of Neurology 62:3, 262-272 (2007).
(49) M. S. Forman, J. Q. Trojanowski, V. M. Y. Lee, Neurodegenerative diseases: a decade of discoveries paves the way for therapeutic breakthroughs. Nature Medicine 10:10, 1055-1063 (2004).
(50) M. M. Zack, R. Kobau, “National and State Estimates of the Numbers of Adults and Children with Active Epilepsy—United States, 2015,” MMWR Morbidity and Mortality Weekly Report 2017 No. 66 (2017).
(51) E. Beghi et al., Global, regional, and national burden of epilepsy, 1990-2016: a systematic analysis for the Global Burden of Disease Study 2016. The Lancet Neurology 18:4, 357-375 (2019).
(52) H. F. Bradford, Glutamate, GABA and epilepsy. Progress in Neurobiology 47:6, 477-511 (1995).
(53) W. Löscher, H. Klitgaard, R. E. Twyman, D. Schmidt, New avenues for anti-epileptic drug discovery and development. Nature Reviews Drug Discovery 12:10, 757-776 (2013).
(54) J. A. French et al., Development of new treatment approaches for epilepsy: Unmet needs and opportunities. Epilepsia 54:Suppl. 4, 3-12 (2013).
(55) R. C. Kessler, W. T. Chiu, O. Demler, E. E. Walters, Prevalence, Severity, and Comorbidity of 12-Month DSM-IV Disorders in the National Comorbidity Survey Replication. Archives of General Psychiatry 62:6, 617-627 (2005).
(56) C. Riaza Bermudo-Soriano, M. M. Perez-Rodriguez, C. Vaquero-Lorenzo, E. Baca-Garcia, New perspectives in glutamate and anxiety. Pharmacology Biochemistry and Behavior 100:4, 752-774 (2012).
(57) B. M. Cortese, K. L. Phan, The Role of Glutamate in Anxiety and Related Disorders. CNS Spectrums 10:10, 820-830 (2005).
(58) J. M. Amiel, S. J. Mathew, Glutamate and anxiety disorders. Current Psychiatry Reports 9:4, 278-283 (2007).
(59) N. W. Schmidt, A. Mishra, J. Wang, W. F. DeGrado, G. C. L. Wong, Influenza Virus A M2 Protein Generates Negative Gaussian Membrane Curvature Necessary for Budding and Scission. Journal of the American Chemical Society 135:37, 13710-13719 (2013).
(60) H. Yao, M. W. Lee, A. J. Waring, G. C. L. Wong, M. Hong, Viral fusion protein transmembrane domain adopts β-strand structure to facilitate membrane topological changes for virus-cell fusion. Proceedings of the National Academy of Sciences 112:35, 10926-10931 (2015).
(61) N. W. Schmidt et al., Criterion for Amino Acid Composition of Defensins and Antimicrobial Peptides Based on Geometry of Membrane Destabilization. Journal of the America Chemical Society 133:17, 6720-6727 (2011).
(62) A. Mishra et al., Translocation of HIV TAT peptide and analogues induced by multiplexed membrane and cytoskeletal interactions. Proceedings of the National Academy of Sciences 108:41, 16883-16888 (2011).
(63) M. W. Lee et al., Molecular Motor Dnm1 Synergistically Induces Membrane Curvature To Facilitate Mitochondrial Fission. ACS Central Science 3:11, 1156-1167 (2017).
(64) M. Marsh, A. Helenius, Virus Entry: Open Sesame. Cell 124:4, 729-740 (2006).
(65) A. E. Smith, A. Helenius, How Viruses Enter Animal Cells. Science 304:5668, 237-242 (2004).
(66) L. Pelkmans, A. Helenius, Insider information: what viruses tell us about endocytosis. Current Opinion in Cell Biology 15:4, 414-422 (2003).
(67) D. S. Dimitrov, Virus entry: molecular mechanisms and biomedical applications. Nature Reviews Microbiology 2:2, 109-122 (2004).
(68) S. Bajaj, D. Dey, R. Bhukar, M. Kumar, M. Banerjee, Non-Enveloped Virus Entry: Structural Determinants and Mechanism of Functioning of a Viral Lytic Peptide. Journal of Molecular Biology 428:17, 3540-3556 (2016).
(69) C. L. Moyer, G. R. Nemerow, Viral weapons of membrane destruction: variable modes of membrane penetration by non-enveloped viruses. Current Opinion in Virology 1:1, 44-49 (2011).
(70) S. Welsch, B. Miller, H.-G. Kräusslich, More than one door—Budding of enveloped viruses through cellular membranes. FEBS Letters 581:11, 2089-2097 (2007).
(71) N. Altan-Bonnet, Y.-H. Chen, Intercellular Transmission of Viral Populations with Vesicles. Journal of Virology 89:24, 12242-12244 (2015).
(72) K. L. Rock, H. Kono, The Inflammatory Response to Cell Death. Annual Review of Pathology: Mechanisms of Disease 3:1, 99-126 (2008).
(73) D. Sumbria, E. Berber, B. T. Rouse, Factors Affecting the Tissue Damaging Consequences of Viral Infections. Frontiers in Microbiology 10:2314, (2019). (74) B. T. Rouse, S. Sehrawat, Immunity and immunopathology to viruses: what decides the outcome? Nature Reviews Immunology 10:7, 514-526 (2010). (75) K. G. Haglid, S. Wang, Y. Qiner, A. Hamberger, Excitotoxicity. Molecular Neurobiology 9:1, 259-263 (1994). (76) J. Van Liefferinge, A. Massie, J. Portelli, G. Di Giovanni, I. Smolders, Are vesicular neurotransmitter transporters potential treatment targets for temporal lobe epilepsy? Frontiers in Cellular Neuroscience 7:139, (2013). The following Example discusses various embodiments of the invention.

### Example 1: Apolipoprotein Mimetic Peptide Inhibits Neutrophil-Driven Inflammatory Damage Via Membrane Remodeling and Suppression of Cell Lysis

Neutrophils are crucial for host defense but are notorious for causing sterile inflammatory damage. Activated neutrophils in inflamed tissue can liberate histone H4, which was recently shown to perpetuate inflammation by permeating membranes via the generation of negative Gaussian curvature (NGC), leading to lytic cell death. Here, we show that it is possible to build peptides or proteins that cancel NGC in membranes and thereby suppress pore formation and demonstrate that they can inhibit H4 membrane remodeling and thereby reduce histone H4-driven lytic cell death and resultant inflammation. As a demonstration of principle, we use apolipoprotein A-I (apoA-I) mimetic peptide apoMP₁. X-ray structural studies and theoretical calculations show that apoMP₁ induces nanoscopic positive Gaussian curvature (PGC), which interacts with the NGC induced by the N-terminus of histone H4 (H4n) to inhibit membrane permeation. Interestingly, we show that induction of PGC can inhibit membrane-permeating activity in general, and “turn off” diverse membrane-permeating molecules besides H4n. In vitro experiments show an apoMP₁ dose-dependent rescue of H4 cytotoxicity. Using a mouse model, we show that tissue accumulation of neutrophils, release of neutrophil extracellular traps (NETs), and extracellular H4 all strongly correlate independently with local tissue cell death in multiple organs, but administration of apoMP₁ inhibits histone H4-mediated cytotoxicity and strongly prevents organ tissue damage.

Although neutrophils play important roles in host defense, they can also precipitate inflammation and tissue damage. Prolonged neutrophil infiltration stimulates inflammation and impedes its resolution.¹ Neutrophil activation triggers the release of inflammatory mediators such as cytokines, granule proteins, and neutrophil extracellular traps (NETs).² Excessive accumulation of such mediators promotes the destruction of the host tissue, resulting in non-resolving pathogenic inflammation. This pathogenic role of neutrophils is exemplified by multiple inflammatory pathologies including neurodegenerative diseases, obesity, arthritis, and sepsis.¹ Recent work indicates that proteins bound to NETs are involved in mediating neutrophil-associated damage. NETs are networks of extracellular fibers composed of DNA and externalized proteins of granule, cytoplasmic, and nuclear origin,³ including histones, the release of which upon cell damage induces tissue damage.^{4,5} A machine-learning classifier⁶ suggested that the N-terminus of histone H4 (H4n) has the capacity to generate negative Gaussian curvature (NGC) in lipid membranes,³ a geometric requirement for membrane-permeating processes such as pore formation,⁷ which can lead to cell death,⁸ itself a potent pro-inflammatory signal. Small-angle X-ray scattering (SAXS) experiments confirmed these predictions.³ Moreover, in hypercholesterolemic mouse models of atherosclerosis,⁹ H4n induced lytic cell death in smooth muscle cells (SMCs), also consistent with machine learning and structural studies. Clearly, regaining control of membrane permeation has important immunological consequences in suppressing runaway neutrophil-driven inflammation.

As disclosed herein, we show conceptually how peptides or proteins that induce positive Gaussian curvature (PGC, dome-shaped surface curvature) can suppress NGC (saddle-shaped surface curvature) and its consequences, including membrane permeation, lytic cell death, and associated organ damage from inflammation using a combination of synchrotron SAXS measurements, mean-field theory, in vitro cell studies, and in vivo animal studies. As a demonstration of principle, we use apoMP₁, a peptide mimic of apolipoprotein A-I (apoA-I), which is a protein that promotes PGC and organizes lipids into compact spherical high-density lipoprotein (HDL) particles for reverse cholesterol transport. Results from X-ray measurements and theoretical studies show that apoMP₁ induces nanoscopic PGC, which interacts with the NGC induced by H4n to inhibit NGC consequences such as membrane permeation. Consistent with this, in vitro experiments using murine endothelial cells, SMCs, macrophages, and hepatocytes show an apoMP₁ dose-dependent rescue of H4 cytotoxicity. Using a mouse model, we show that tissue accumulation of neutrophils, release of NETs, and extracellular H4 all strongly correlate independently with local tissue cell death in multiple organs, but administration of apoMP₁ inhibits histone H4-mediated cytotoxicity and strongly prevents organ tissue damage. These results exemplify a general mechanism for the suppression of membrane permeation that is mediated by membrane curvature rather than allosteric binding. The generality of these membrane-mediated interactions is highlighted with apoMP₁'s ability to “turn off” the membrane-remodeling activities of antimicrobial peptides (AMPs) and cell-penetrating peptides (CPPs), which are known to generate NGC in lipid membranes.^{7, 10-14} To show that these effects are not limited to apolipoprotein-like peptides and to validate this general concept of canceling out induced NGC by applying PGC, we design a peptide and demonstrate that it too is able to suppress the effects of membrane-disruptive peptides. Taken together, these results imply that histone-mediated cell death can be modulated with greater precision and resolution than previous thought possible through controlled membrane remodeling. More notably, the application of curvature as a means to non-specifically inhibit membrane-remodeling events will help inform the development of therapeutics to treat conditions such as chronic inflammation.

The generation of NGC by pore-forming peptides is well known.^{7,10-12} To study the interaction between induced NGC and PGC in membranes, we need peptide sequences that (1) can generate nanoscopic positive curvature and (2) are soluble and able to freely exchange between different membranes. Lipoproteins are macromolecular complexes that consist of a hydrophobic core of neutral lipids, such as triglycerides and cholesteryl esters, surrounded by an outer layer of phospholipids, free cholesterol, and proteins. Lipoprotein particles are grouped into four major classes based on their density and size: chylomicrons, very low-density lipoproteins (VLDL), low-density lipoproteins (LDL), and HDL. Apolipoproteins, the protein component of lipoproteins, stabilize the geometric structure of lipoprotein particles as they undergo compositional modifications and are broadly classified into two groups, exchangeable (water-soluble) apolipoproteins, which can transfer between lipoprotein particles, and non-exchangeable (water-insoluble) apolipoproteins, which remain bound to the same lipoprotein particle from biosynthesis to breakdown. The transport of lipids is typically facilitated by exchangeable apolipoproteins due to their ability to reversibly associate and dissociate from lipoproteins.¹⁵⁻¹⁸ Such exchangeable apolipoproteins are composed of multiple amphipathic α-helices,^{19, 20} which have been grouped into taxonomies based on their physicochemical properties.^{15, 21} While the distribution of different classes of amphipathic helices varies both within and among the different apolipoproteins, and is believed to account for their diverse lipid affinities and functional properties,^{22, 23} details of how such helices enforce and maintain distinct lipoprotein structures are not known.

ApoA-I, an exchangeable apolipoprotein that contains ten amphipathic α-helical segments, is the major protein constituent of HDL particles^{24, 25} and is central to HDL generation.²⁶ ApoA-I assembles lipids into spherical HDL species that are 7-12 nm in diameter.^{24, 27} When mixed, apoA-I and phospholipids are shown to spontaneously associate to form small micelle-like protein-lipid complexes.^{15, 28, 29} Taken together, these findings suggest that the physicochemical properties and characteristics of apoA-I are conducive to the generation of PGC, although it is not clear a priori whether the quantitative amount of induced PGC is sufficient to turn off NGC needed for pore formation. Moreover, apoA-I being an exchangeable apolipoprotein allows for its diffusion and recycling between different cell membranes. Peptide analogs have been designed to mimic the lipid-associating helices of apoA-I.³⁰⁻³⁴ Despite not having sequence homology to apoA-I, these mimetic peptides have individually been shown to recapitulate different aspects of apoA-I properties,³⁵⁻³⁷ including, interestingly, some anti-inflammatory properties.³⁸ However, given the molecular differences between apoA-I and its peptide analogs and among the peptide analogs, the molecular basis of their therapeutic effects in dampening inflammation is not clear. Based on these considerations, we choose a well-characterized apolipoprotein A-I mimetic peptide (apoMP) D-W-F-K-A-F-Y-D-K-V-A-E-K-F-K-E-A-F^{30, 39} (SEQ ID NO: 2) (D4F, which we refer to herein as apoMP₁ to emphasize its origin and functional role in self-assembly of compact spherical lipid particles).

### Results/Discussion

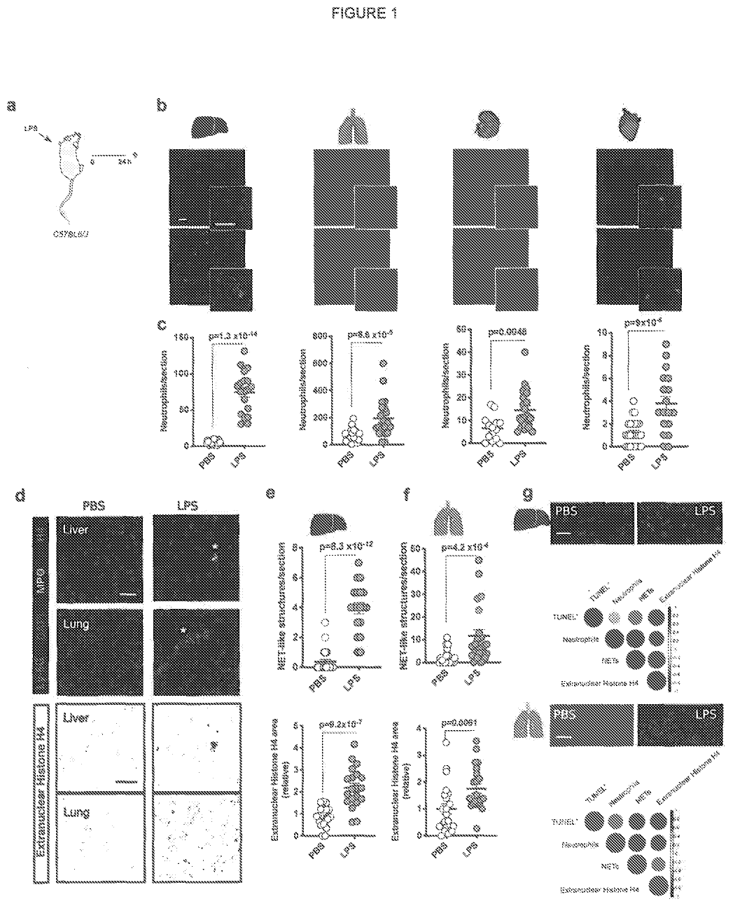

Uncontrolled inflammatory responses that occur during systemic inflammation can cause tissue destruction, multi-organ failure, and death.⁴⁰ Neutrophil hyperactivation in response to bacterial products triggers the release of NETs, the accumulation of which is associated with mortality during septicemia.⁴ To interrogate the importance of NET-derived histone H4 on organ tissue damage during systemic inflammation, we administered lipopolysaccharide (LPS) from E. coli (O111:B4) intravenously in mice to mimic systemic Gram-negative bacterial infection (FIG. 1a). LPS-induced endotoxemia associated with neutrophil activation and triggered multi-organ neutrophil infiltration (FIG. 1b,c). In particular, in organs such as the lung and liver, which are especially vulnerable during endotoxemia, LPS infusion induced NET release and the accumulation of extranuclear histone H4 (FIG. 1d-f), both positively associated with increased tissue cell death (FIG. 1g). Importantly, the levels of extranuclear histone H4 in the tissue positively correlated with the number of infiltrated neutrophils, hence suggesting that tissue histone H4 is of neutrophil origin. Collectively, systemic activation of circulating neutrophils results in their entrapment in multiple tissues and the release of NET-derived histone H4 to cause tissue damage.

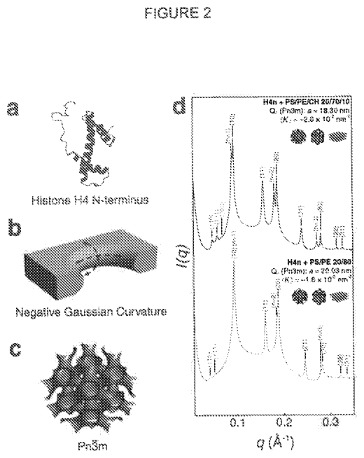

Through mouse models of endotoxemia, we demonstrated that histone H4 molecules released during NETosis induce tissue damage and cell death in multiple organ systems. In previous studies, we showed that H4n can directly interact with cell membranes, remodel membrane curvature, and promote lytic cell death.³ H4n consists of 24 amino acids, which include two α-helical segments in the α1 domain of the full histone H4 protein (FIG. 2a). Its capacity to form membrane pores has been attributed to the ability of its N-terminal domain to induce NGC in cell membranes, which is a geometric requirement for membrane-permeating events. Curvature at a point on a surface can be described by its principal curvatures, c₁ and c₂, along orthogonal axes. For a saddle-shaped deformation, the surface bends in opposite directions along these axes (c₁>0, c₂<0), resulting in a Gaussian curvature, K≡c₁c₂, that is negative (FIG. 2b). These saddle-shaped surfaces are required to form the inner surface of a transmembrane pore and at the bases of blebs.

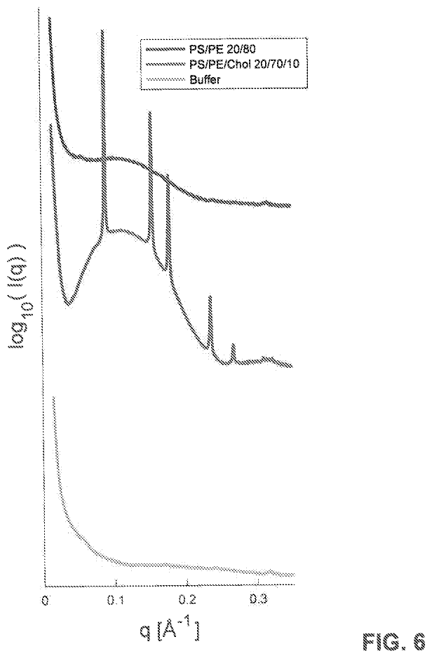

Before assessing how apoMP₁ may influence the pore-forming activity of H4n, we conducted a control study to confirm H4n's ability to generate membrane-destabilizing curvature at lipid compositions of interest. Small unilamellar vesicles (SUVs) were prepared from ternary lipid mixtures of phosphatidylserine (PS), phosphatidylethanolamine (PE), and cholesterol (CH) at molar ratios of PS/PE 20/80 and PS/PE/CH 20/70/10. Because cholesterol constitutes a large fraction of mammalian cell membranes,⁴¹ we used physiologically relevant cholesterol-containing SUVs to examine the effect of cholesterol on the membrane-remodeling properties of H4n. For the model membrane PS/PE 20/80, we observed a coexistence of three phases: (1) a lamellar phase (L_{α}) (peaks with q-ratios 1:2:3), (2) an inverse hexagonal phase (H_{II}) (peaks with q-ratios √1:√3:√4:√7:√9), and (3) a Pn3m inverse bicontinuous cubic phase (Q_{II}) with a lattice parameter of 20.03 nm (peaks with q-ratios √2:√3:√4) (FIG. 2d). The Pn3m phase is rich in NGC (FIG. 2c). For the cholesterol-containing model membrane PS/PE/CH 20/70/10, we again observed a similar phase coexistence, but with a smaller lattice parameter in the Pn3m phase (18.30 nm) (FIG. 2d, Table S1). In comparison, the control samples of PS/PE 20/80 SUVs only showed a broad feature of unilamellar vesicles, and the control samples of PS/PE/CH 20/70/10 SUVs only exhibited an Hi phase (FIG. 6). To compare the Pn3m phases between cholesterol-rich and cholesterol-poor systems, we calculated the average amount of Gaussian curvature generated in each system using the equation (K)=2πχ/A₀a², where a is the lattice parameter The Euler characteristic, χ, and the surface area per unit cell, A₀, are specific for each cubic phase. For Pn3m, χ=−2 and A₀=1.919. An increase in NGC magnitude from (K)=−1.6×10⁻² nm² for the PS/PE 20/80 membrane to (K)=−2.0×10⁻² nm² for the PS/PE/CH 20/70/10 membrane indicates that the presence of cholesterol promotes the ability of the H4n to generate membrane curvature. Interestingly, the magnitudes of NGC measured here are comparable with those generated by more hydrophobic AMPs with non-specific activity against eukaryotic cells,⁴² consistent with the contention of lytic cell death, and within range of previous measurements for H4n.³ Moreover, SAXS measurements indicate that H4n's ability to generate membrane-permeating curvature and lytic cell death increases with cholesterol content, which typically constitutes 30%-40%⁴¹ of mammalian cell membranes.

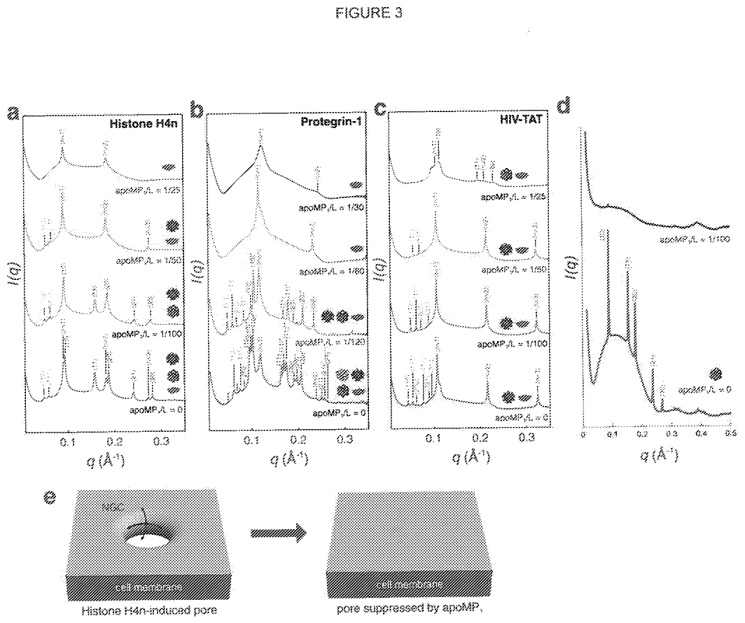

We hypothesized that apoMPs can interact with membranes in a manner that interferes with the membrane activity of histone H4, and consequently, attenuate neutrophil-driven cytotoxicity and alleviate associated inflammation. We therefore investigated in detail the effect of apoMP₁ on NGC generation by H4n. We incubated PS/PE/CH 20/70/10 SUVs with H4n at a peptide-to-lipid (P/L) molar ratio of 1/40 and apoMP₁ at P/L molar ratios of 1/100, 1/50, and 1/25. SAXS measurements had shown that H4n alone induced correlation peaks characteristic of coexisting L_{α}. H_{II}, and Pn3m Q_{II} phases rich in NGC (FIG. 2d). When co-administered with apoMP₁, however, the NGC-rich Pn3m cubic phases generated by H4n gradually disappeared with increasing concentration of apoMP₁ (FIG. 3a, Table S1). These results imply that apoMP₁ can suppress effects associated with NGC generation (FIG. 3e).

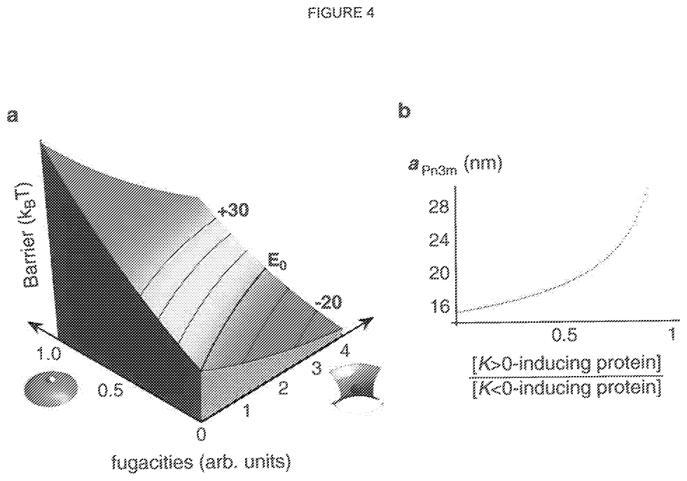

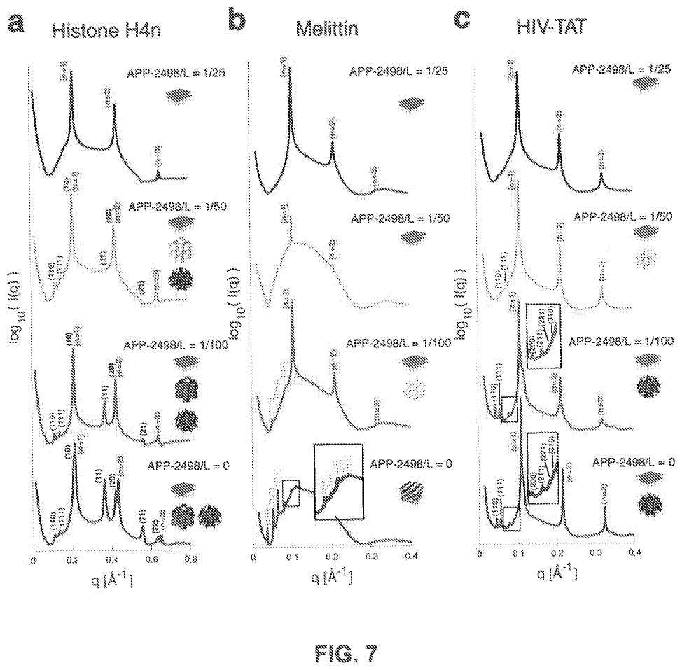

Interestingly, given that apolipoproteins are not known to bind histones, our results suggest that H4n and apoMP₁ interact with one another via a different mechanism rather than direct binding and allosteric inhibition. Since apoA-I assembles spheroidal HDL particles with small radii, it can stabilize positive membrane curvature by definition. Furthermore, deleting the helices that have the highest lipid affinities causes apoA-I to form larger particles, which have less positive curvature.⁴³ Consistent with that expectation, we find experimentally that apoMP₁ can transform an inverse hexagonal phase with strong negative mean curvature into a lamellar phase with zero mean curvature (FIG. 3d). To test the idea that an apoMP may suppress H4n pore-forming activity via membrane-mediated interactions, we developed a theoretical model (Supporting Information) for mixtures of two membrane-bound peptides (or proteins), one inducing negative (−) Gaussian curvature (ex: saddle-shaped deformations from H4n) and the other positive (+) Gaussian curvature (ex: dome-shaped deformations from apoMP₁), in which positive curvature is induced in the membrane with no assumed preferred direction. Each peptide is taken to induce local principal curvatures in the membrane, ξ₁ and ξ₂, which work against the membrane deformations given by the Helfrich free energy over the membrane area FHelfrich=(π/2)∫dA(a₂+a₂)² with bending rigidity κ. The energy of a single (±) peptide is E₂/(k_{B}T)≡(λ/2)[(a₁−ξ_{1,z})²+(a₂−ξ_{2,z})²] when the peptide's maximal curvature is aligned with the membranes, where (λ/2)+(ξ_{1, ±}²+ξ_{2, ±}²) is the energy cost of binding a peptide to a flat membrane, λ is a dimensionless coupling constant, and the induced principal curvatures depend on the peptide type. Analogous to classical nucleation theory, the free energy scales as E=−σπΓ for a pore of radius r and pore rim line tension Γ on a membrane with effective surface tension σ. This model exhibits an energy barrier E_{barrier}=πΓ²/σ at a radius r₀=Γ/σ. When this energy is comparable to thermal energy, pore formation becomes favorable. Mixtures of peptides inducing competing curvatures can control pore formation via this free energy barrier. We find that K<0-inducing peptides, such as H4n, universally suppress the free energy barrier, while peptides that induce PGC (K>0), such as apoMP₁, raise the barrier drastically even at modest area fractions (FIG. 4a). The precise values used for constants do not change the generic behavior observed in the model. Additionally, the model predicts that the cubic phase lattice parameter a_{Pn3m} generally grows with increasing K>0 to K<0-inducing peptide ratio (FIG. 4b). (To see a summary of the interplay between Gaussian curvature, Gaussian modulus κ̅, and the propensity for pore formation, we refer the interested reader to a recent review.⁴⁴) The suppression mechanism of membrane permeation described by the above theoretical model does not require specific binding between the two types of peptides. To investigate the generality of apoMP₁-induced NGC suppression, we examined whether apoMP₁ could turn off NGC generated by amphiphilic peptides unrelated to histone-induced inflammation and with no sequence homology to histones, including the AMP protegrin-1 (PG-1) and the CPP human immunodeficiency virus type 1 trans-activator of transcription (HIV-TAT). PG-1 is an 18-amino acid β-hairpin structure⁴⁵ and possesses broad-spectrum antimicrobial activity against bacteria, fungi, and viruses⁴⁶ via membrane disruption.⁴⁷ We incubated PS/PE 20/80 SUVs with PG-1 at a P/L molar ratio of 1/40. PG-1 alone induced correlation peaks characteristic of coexisting L_{α}, H_{II}, Pn3m Q_{II}, and Im3m Q_{II} phases (FIG. 3b, Table S1). ApoMP₁ exhibited dose-dependent suppression of the NGC induced by PG-1: apoMP₁ first suppressed the Im3m phase at a P/L ratio of 1/120 before completely eliminating NGC, as evidenced by the absence of Pn3m phase peaks at a P/L ratio of 1/60. Similarly, apoMP₁ suppresses NGC generated by the CPP HIV-TAT,¹⁴ a protein that enhances viral transcription.⁴⁸ HIV-TAT alone at a P/L ratio of 1/40 remodeled PS/PE 20/80 SUVs into L_{α} and Pn3m Q_{II} phases. In the presence of apoMP₁, at increasing P/L ratios from 1/100 to 1/25, NGC was again suppressed in a dose-dependent manner. At the highest P/L ratio for apoMPs, the system showed a complete absence of NGC-rich cubic phases (FIG. 3c, Table S1). Clearly, apoMP₁ was able to suppress NGC generation, not just for H4n, but across disparate membrane-remodeling amphiphilic peptides that span AMPs and CPPs, which are drastically different from H4n in sequence, charge, and hydrophobicity. Certainly, it is possible for pore inhibition to involve direct binding between a pore-inhibiting peptide (or protein) and a pore-forming peptide (or protein),⁴⁹ based on their individual attributes. However, the results here demonstrating that a single peptide can suppress the effects of multiple pore-forming peptides indicate that direct binding is not required to inhibit the activity of a pore former, and thus, suggests that a general, non-specific mechanism exists in parallel. In fact, the generality of these interactions mediated by induced membrane curvature can be seen in extant work on apoMPs: Despite their sequence diversity and lack of sequence homology to apoA-I, a vast number of apoMPs have successfully recapitulated many of the biological functions of apoA-I by mimicking its physicochemical and structural properties. To emphasize this point and the potential to multiplex different functions into these PGC-generating peptide sequences, we designed an anti-pore peptide APP-2498 that primarily uses arginine residues for its positively charged residues. In contrast, nearly all studied apoMPs exclusively utilize lysine in their positively charged residue positions.^{22, 31, 33, 34, 39, 50-52} We demonstrate that APP-2498 is capable of similar NGC suppression as apoMP₁ (Table S2, FIG. 7). Indeed, we expect the existence of a general design architecture for anti-pore peptides.

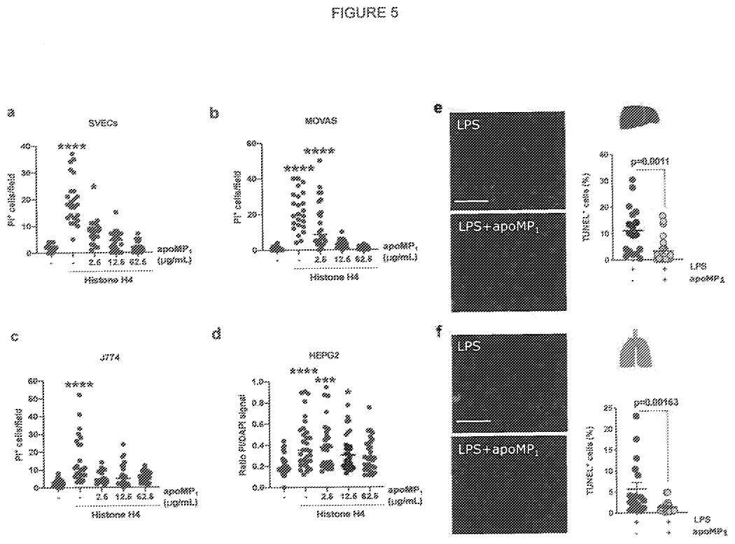

SAXS measurements indicate that in the presence of apoMP₁, the generation of NGC by H4n is greatly attenuated, suggesting that the peptide is able to inhibit membrane-lytic cell death. Consistent with these observations, apoMP₁ was able to strongly inhibit the toxic capacity of H4n in different cell types in a dose-dependent fashion (FIG. 5a-d). These effects were pronounced in endothelial cells, smooth muscle cells, and macrophages, while minor, yet significant, for hepatocytes. Next, to explore the therapeutic ability of apoMP₁ to prevent histone H4-mediated tissue damage, we treated mice with a single dose of apoMP₁ together with LPS administration. Notably, apoMP₁ treatment fully abrogated liver (FIG. 5e) and lung (FIG. 5f) tissue damage caused by LPS-induced hyperinflammation. Overall, our results support a host-tissue protective function of apoA-I through the prevention of NGC generation by neutrophil-derived histone H4.

Placing our results in a broader context, existing studies have shown that administration of either HDL or its primary protein component, apoA-I, exhibit anti-inflammatory and protective effects, including the reduction of atherosclerosis,⁵³ sepsis,^{54, 55} tumor growth,⁵⁶ macrophage activation,⁵⁷ and pro-inflammatory cytokine production.⁵⁸ However, due to the high dosages of apoA-I needed to achieve these effects, the design of smaller synthetic peptides to recapitulate the activities of apoA-I has recently attracted attention.^{59, 60} Both animal and human studies have shown that administration of these mimetic peptides leads to similar anti-inflammatory effects, which can treat conditions such as renal⁶¹ and hepatic⁶² inflammation, insulin resistance and diabetes,³⁶ arthritis,⁶³ asthma,⁶⁴ acute vascular inflammation,³¹ and atherosclerosis.^{30, 33, 35, 65-68} Despite the ability of these mimetic designs to replicate the activities of HDL and apoA-I, the detailed mechanisms of their shared anti-inflammatory activities have been unclear. The findings presented here now suggest that inhibition of NGC generation in membranes by apoA-I and its mimics may explain some their observed anti-inflammatory effects.

### Conclusions

A major challenge in modern medicine is discovering effective therapeutic strategies to inhibit cell death associated with inflammation, which impacts tissue function even after resolution of the acute inflammatory event. We show that LPS-induced sepsis causes neutrophil hyperactivation and release of NETs containing externalized nuclear proteins, including histone H4. Results here indicate that H4n induces rapid, receptor-independent cell death in multiple organs by generating NGC in cell membranes and forming membrane pores, in a manner that is promoted by cholesterol. Importantly, our findings suggest that an apoA-I mimetic peptide has the capacity to suppress histone-induced pore formation and cell death, and thereby prevent systematic tissue damage, through a membrane-mediated mechanism that is independent from direct protein-protein interactions. Our findings here showing that peptides that can suppress NGC have the ability to prevent systemic tissue and organ damage thus carry significant implications for the treatment of a broad range of inflammatory conditions.

### Methods/Experimental

Complete details of the experimental materials and methods are provided in the Supporting Information below.

Peptides. Peptide apoMP₁ was a generous gift from Dr. Alan Fogelman. All other peptides were synthesized using solid-phase synthesis by LifeTein or purchased from Anaspec.

SAXS experiments. Lyophilized phospholipids were purchased from Avanti Polar Lipids and dissolved in chloroform as individual stock solutions. Lipid mixtures were prepared by combining the lipid stock solutions at the desired molar ratios and subsequently evaporated under nitrogen and desiccated overnight. The resulting dry lipid films were resuspended in aqueous buffer solution and incubated overnight at 37° C. Lipid suspensions were sonicated until clear and extruded through a 0.2 μm pore filter to form SUVs. SUVs were mixed with peptides at specified P/L molar ratios and characterized using SAXS at the Stanford Synchrotron Radiation Lightsource (SSRL) and the Advanced Light Source (ALS).

Animal experiments. All mouse experiments were performed according to European guidelines for Care and Use of laboratory Animals. Protocols were approved by the Committee on the Ethics of Animal Experiments of the Regierung von Oberbayern. For the mouse endotoxemia model, C57BL6/J mice were challenged with 10 mg/kg of LPS from E. Coli (0111:B4) and treated with 250 μg apoMP₁ intraperitoneally or saline as control. Mice were sacrificed after 24 hours and lung, liver, kidney, and heart tissues were isolated and processed. Immunofluorescence staining was performed on fixed cryosections and imaged using confocal microscopy.

Cell viability assays. Mouse vascular aorta/smooth muscle cells (MOVAS), J774A.1 macrophages, SVEC4-10 endothelial cells, and HepG2 human hepatocytes were incubated with 50 μg/mL of histone H4 (Biomol) and specified amounts of apoMP₁. Cell viability was determined by propidium iodide (PI) uptake.

Statistics. Statistical analysis was performed by GraphPad Prism 7 software (GraphPad Software). The ROUT outlier function was used to exclude statistical outliers (Q=1%). Normal distribution of the data was assessed using the D'Agostino-Pearson omnibus test for normality. Normally distributed data was tested by two-tailed unpaired t-test (one variable) or one-way ANOVA with Tukey's or Dunnet's correction (>2 variables). When 2 factors were analyzed, data was analyzed using two-way ANOVA with Tukey's correction. In all tests a 95% confidence interval was used, with which p<0.05 was considered a significant difference. Correlograms were generated using R. All data is represented as mean±SEM.

### Example 1 References

1. Soehnlein, O.; Steffens, S.; Hidalgo, A.; Weber, C. Neutrophils As Protagonists and Targets in Chronic Inflammation. Nat. Rev. Immunol. 2017, 17, 248-261.
2. Papayannopoulos, V. Neutrophil Extracellular Traps in Immunity and Disease. Nat. Rev. Immunol. 2018, 18, 134-147.
3. Silvestre-Roig, C.; Braster, Q.; Wichapong, K.; Lee, E. Y.; Teulon, J. M.; Berrebeh, N.; Winter, J.; Adrover, J. M.; Santos, G. S.; Froese, A.; Lemnitzer, P.; Ortega-Gómez, A.; Chevre, R.; Marschner, J.; Schumski, A.; Winter, C.; Perez-Olivares, L.; Pan, C.; Paulin, N.; Schoufour, T.; et al. Externalized Histone H4 Orchestrates Chronic Inflammation by Inducing Lytic Cell Death. Nature 2019, 569, 236-240.
4. Xu, J.; Zhang, X.; Pelayo, R.; Monestier, M.; Ammollo, C. T.; Semeraro, F.; Taylor, F. B.: Esmon, N. L.: Lupu, F.: Esmon, C. T. Extracellular Histones Are Major Mediators of Death in Sepsis. Nat. Med. 2009, 15, 1318-1321.
5. Saffarzadeh, M.; Juenemann, C.; Queisser, M. A.; Lochnit, G.; Barreto, G.; Galuska, S. P.; Lohmeyer, J.; Preissner, K. T. Neutrophil Extracellular Traps Directly Induce Epithelial and Endothelial Cell Death: A Predominant Role of Histones. PLoS One 2012, 7, e32366.
6. Lee, E. Y.; Fulan, B. M.; Wong, G. C. L.; Ferguson, A. L. Mapping Membrane Activity in Undiscovered Peptide Sequence Space Using Machine Learning. Proc. Natl. Acad. Sci. U.S.A 2016, 113, 13588-13593.
7. Schmidt, N. W.; Mishra, A.; Lai, G. H.; Davis, M.; Sanders, L. K.; Tran, D.; Garcia, A.; Tai, K. P.; McCray, P. B.; Ouellette, A. J.; Selsted, M. E.; Wong, G. C. L. Criterion for Amino Acid Composition of Defensins and Antimicrobial Peptides Based on Geometry of Membrane Destabilization. J. Am. Chem. Soc. 2011, 133, 6720-6727.
8. Dathe, M.; Wieprecht, T. Structural Features of Helical Antimicrobial Peptides: Their Potential to Modulate Activity on Model Membranes and Biological Cells. Biochim. Biophys. Acta 1999, 1462, 71-87.
9. Silvestre-Roig, C.; Winther, M. P. d.; Weber, C.; Daemen, M. J.; Lutgens, E.; Soehnlein, O. Atherosclerotic Plaque Destabilization. Circul. Res. 2014, 114, 214-226.
10. Hickel, A.; Danner-Pongratz, S.; Amenitsch, H.; Degovics, G.: Rappolt, M.; Lohner, K.; Pabst, G. Influence of Antimicrobial Peptides on the Formation of Nonlamellar Lipid Mesophases. Biochim. Biophys. Acta 2008, 1778, 2325-2333.
11. Prenner, E. J.; Lewis, R. N. A. H.; Neuman, K. C.; Gruner, S. M.; Kondejewski, L. H.; Hodges, R. S.; McElhaney, R. N. Nonlamellar Phases Induced by the Interaction of Gramicidin S with Lipid Bilayers. A Possible Relationship to Membrane-Disrupting Activity. Biochemistry 1997, 36, 7906-7916.
12. Keller, S. L.; Gruner, S. M.; Gawrisch, K. Small Concentrations of Alamethicin Induce a Cubic Phase in Bulk Phosphatidylethanolamine Mixtures. Biochim. Biophys. Acta 1996, 1278, 241-246.
13. Schmidt, N.; Mishra, A.; Lai, G. H.; Wong, G. C. L. Arginine-Rich Cell-Penetrating Peptides. FEBS Lett. 2010, 584, 1806-1813.
14. Mishra, A.; Lai, G. H.; Schmidt, N. W.; Sun, V. Z.; Rodriguez, A. R.; Tong, R.;
Tang, L.; Cheng, J.; Deming, T. J.; Kamei, D. T.; Wong, G. C. L. Translocation of HIV TAT Peptide and Analogues Induced by Multiplexed Membrane and Cytoskeletal Interactions. Proc. Natl. Acad. Sci. U.S.A 2011, 108, 16883-16888.
15. Segrest, J. P.; Jones, M. K.; De Loof, H.; Brouillette, C. G.; Venkatachalapathi, Y. V.; Anantharamaiah, G. M. The Amphipathic Helix in the Exchangeable Apolipoproteins: A Review of Secondary Structure and Function. J. Lipid Res. 1992, 33, 141-166.
16. Remaley, A. T.; Schumacher, U. K.; Stonik, J. A.; Farsi, B. D.; Nazih, H.; Brewer, H. B. Decreased Reverse Cholesterol Transport from Tangier Disease Fibroblasts. Atertio. Thromb. Vasc. Biol. 1997, 17, 1813-1821.
17. Remaley, A. T.; Stonik, J. A.; Demosky, S. J.; Neufeld, E. B.; Bocharov, A. V.; Vishnyakova, T. G.; Eggerman, T. L., Patterson, A. P.; Duverger, N. J.; Santamarina-Fojo, S.; Brewer, H. B. Apolipoprotein Specificity for Lipid Efflux by the Human ABCAI Transporter. Biochem. Biophys. Res. Commun. 2001, 280, 818-823.
18. Smith, L. E.; Segrest, J. P.; Davidson, W. S. Helical Domains That Mediate Lipid Solubilization and ABCA1-Specific Cholesterol Efflux in Apolipoproteins C-I and A-II. J. Lipid Res. 2013, 54, 1939-1948.
19. McLachlan, A. D. Repeated Helical Pattern in Apolipoprotein-A-I. Nature 1977, 267, 465-466.
20. Li, W. H.; Tanimura, M.; Luo, C. C.; Datta, S.; Chan, L. The Apolipoprotein Multigene Family: Biosynthesis, Structure, Structure-Function Relationships, and Evolution. J. Lipid Res. 1988, 29, 245-271.
21. Segrest, J. P.; De Loof, H.; Dohlman, J. G.; Brouillette, C. G.; Anantharamaiah, G. M. Amphipathic Helix Motif: Classes and Properties. Proteins: Struct. Funct. Bioinform. 1990, 8, 103-117.
22. Mishra, V. K.; Palgunachari, M. N. Interaction of Model Class A1, Class A2, and Class Y Amphipathic Helical Peptides with Membranes. Biochemistry 1996, 35, 11210-11220.
23. Saito, H.; Lund-Katz, S.; Phillips, M. C. Contributions of Domain Structure and Lipid Interaction to the Functionality of Exchangeable Human Apolipoproteins. Prog. Lipid Res. 2004, 43, 350-380.
24. Davidson, W. S.; Thompson, T. B. The Structure of Apolipoprotein A-I in High Density Lipoproteins. J. Biol. Chem. 2007, 282, 22249-22253.
25. Kontush, A.; Chapman, M. J. High-Density Lipoproteins: Structure, Metabolism, Function, and Therapeutics. John Wiley & Sons, Inc.: Hoboken, New Jersey, USA, 2011; p 648.
26. Mishra, V. K.; Palgunachari, M. N.; Datta, G.; Phillips, M. C.; Lund-Katz, S.; Adeyeye, S. O.; Segrest, J. P.; Anantharamaiah, G. M. Studies of Synthetic Peptides of Human Apolipoprotein A-I Containing Tandem Amphipathic α-Helixes. Biochemistry 1998, 37, 10313-10324.
27. Silva, R. A. G. D.; Huang, R.; Morris, J.; Fang, J.; Gracheva, E. O.; Ren, G.; Kontush, A.; Jerome, W. G.; Rye, K.-A.; Davidson, W. S. Structure of Apolipoprotein A-I in Spherical High Density Lipoproteins of Different Sizes. Proc. Natl. Acad. Sci. U.S.A. 2008, 105, 12176-12181.
28. Tall, A. R.; Small, D. M.; Shipley, G. G.; Lees, R. S. Apoprotein Stability and Lipid-Protein Interactions in Human Plasma High Density Lipoproteins. Proc. Natl. Acad. Sci. U.S.A 1975, 72, 4940-4942.
29. Atkinson, D.; Smith, H. M.; Dickson, J., Austin, J. P. Interaction of Apoprotein from Porcine High-Density Lipoprotein with Dimyristoyl Lecithin. Eur. J. Biochem. 1976, 64, 541-547.
30. Navab, M., Anantharamaiah, G. M., Reddy, S. T.; Hama, S.; Hough, G.; Grijalva, V. R.; Yu, N.; Ansell, B. J.; Datta, G.; Garber, D. W.; Fogelman, A. M. Apolipoprotein A-I Mimetic Peptides. Atertio. Thromb. Vasc. Biol. 2005, 25, 1325-1331.
31. Di Bartolo, B. A.; Nicholls, S. J.; Bao, S.; Rye, K.-A.; Heather, A. K.; Barter, P. J.; Bursill, C. The Apolipoprotein A-I Mimetic Peptide ETC-642 Exhibits Anti-Inflammatory Properties That Are Comparable to High Density Lipoproteins. Atherosclerosis 2011, 217, 395-400.
32. Wool, G. D.; Reardon, C. A.; Getz, G. S. Apolipoprotein A-I Mimetic Peptide Helix Number and Helix Linker Influence Potentially Anti-Atherogenic Properties. J. Lipid Res. 2008, 49, 1268-1283.
33. D'Souza, W.; Stonik, J. A.; Murphy, A.; Demosky, S. J.; Sethi, A. A.; Moore, X. L.; Chin-Dusting, J.; Remaley, A. T.; Sviridov, D. Structure/Function Relationships of Apolipoprotein A-I Mimetic Peptides. Circul. Res. 2010, 107, 217-227.
34. Mendez, A. J.; Anantharamaiah, G. M.; Segrest, J. P.; Oram, J. F. Synthetic Amphipathic Helical Peptides That Mimic Apolipoprotein A-I in Clearing Cellular Cholesterol. J. Clin. Invest. 1994, 94, 1698-1705.
35. Garber, D. W.; Datta, G.; Chaddha, M.; Palgunachari, M. N.; Hama, S. Y.; Navab, M.; Fogelman, A. M.; Segrest, J. P.; Anantharamaiah, G. M. A New Synthetic Class A Amphipathic Peptide Analogue Protects Mice from Diet-Induced Atherosclerosis. J. Lipid Res. 2001, 42, 545-552.
36. Morgantini, C.; Imaizumi, S.; Grijalva, V.; Navab, M.; Fogelman, A. M.; Reddy, S. T. Apolipoprotein A-I Mimetic Peptides Prevent Atherosclerosis Development and Reduce Plaque Inflammation in a Murine Model of Diabetes. Diabetes 2010, 59, 3223-3228.
37. Van Lenten, B. J.; Wagner, A. C.; Anantharamaiah, G. M.; Navab, M.; Reddy, S. T.; Buga, G. M.; Fogelman, A. M. Apolipoprotein A-I Mimetic Peptides. Curr. Atheroscler. Rep. 2009, 11, 52-57.
38. Anantharamaiah, G. M.; Mishra, V. K.; Garber, D. W.; Datta, G.; Handattu, S. P.; Palgunachari, M. N.; Chaddha, M.; Navab, M., Reddy, S. T.; Segrest, J. P.; Fogelman, A. M. Structural Requirements for Antioxidative and Anti-Inflammatory Properties of Apolipoprotein A-I Mimetic Peptides. J. Lipid Res. 2007, 48, 1915-1923.
39. Datta, G.; Chaddha, M.; Hama, S.; Navab, M.; Fogelman, A. M.; Garber, D. W., Mishra, V. K.; Epand, R. M.; Epand, R. F.; Lund-Katz, S.; Phillips, M. C.; Segrest, J. P.; Anantharamaiah, G. M. Effects of Increasing Hydrophobicity on the Physical-Chemical and Biological Properties of a Class A Amphipathic Helical Peptide. J. Lipid Res. 2001, 42, 1096-1104.
40. Angus, D. C.; van der Poll, T. Severe Sepsis and Septic Shock. New Engl. J. Med. 2013, 369, 840-851.
41. Pinkwart, K.; Schneider, F.; Lukoseviciute, M.; Sauka-Spengler, T.; Lyman, E.; Eggeling, C.; Sezgin, E. Nanoscale Dynamics of Cholesterol in the Cell Membrane. J. Biol. Chem. 2019, 294, 12599-12609.
42. Lee, M. W.; de Anda, J.; Kroll, C.; Bieniossek, C.; Bradley, K.; Amrein, K. E.; Wong, G. C. L. How Do Cyclic Antibiotics with Activity against Gram-Negative Bacteria Permeate Membranes? A Machine Learning Informed Experimental Study. Biochim. Biophys. Acta 2020, 1862, 183302.
43. Vedhachalam, C.; Chetty, P. S.; Nickel, M.; Dhanasekaran, P.; Lund-Katz, S.; Rothblat, G. H.; Phillips, M. C. Influence of Apolipoprotein (Apo) A-I Structure on Nascent High Density Lipoprotein (HDL) Particle Size Distribution. J. Biol. Chem. 2010, 285, 31965-31973.
44. Schmidt, N. W.; Wong, G. C. L. Antimicrobial Peptides and Induced Membrane Curvature: Geometry, Coordination Chemistry, and Molecular Engineering. Curr. Opin. Solid State Mater. Sci. 2013, 17, 151-163.
45. Steinberg, D. A.; Hurst, M. A.; Fujii, C. A.; Kung, A. H.; Ho, J. F.; Cheng, F. C.; Loury, D. J.; Fiddes, J. C. Protegrin-1: A Broad-Spectrum, Rapidly Microbicidal Peptide with in Vivo Activity. Antimicrob. Agents Chemother. 1997, 4/, 1738-1742.
46. Kokryakov, V. N.; Harwig, S. S. L.; Panyutich, E. A.; Shevchenko, A. A.; Aleshina, G. M.; Shamova, O. V.; Korneva, H. A.; Lehrer, R. 1. Protegrins: Leukocyte Antimicrobial Peptides That Combine Features of Corticostatic Defensins and Tachyplesins. FEBS Lett. 1993, 327, 231-236.
47. Lam, K. L. H.; Ishitsuka, Y.; Cheng, Y.; Chien, K.; Waring, A. J.; Lehrer, R. I.; Lee, K. Y. C. Mechanism of Supported Membrane Disruption by Antimicrobial Peptide Protegrin-1. J. Phys. Chem. B 2006, 110, 21282-21286.
48. Debaisieux, S.; Rayne, F.; Yezid, H.; Beaumelle, B. The Ins and Outs of HIV-1 TAT. Traffic 2012, 13, 355-363.
49. Wang, Y.; Agerberth, B.; Lothgren, A.; Almstedt, A.; Johansson, J. Apolipoprotein A-I Binds and Inhibits the Human Antibacterial/Cytotoxic Peptide LL-37. J. Biol. Chem. 1998, 273, 33115-33118.
50. Sethi, A. A.; Stonik, J. A.; Thomas, F.; Demosky, S. J.; Amar, M.; Neufeld, E.; Brewer, H. B.; Davidson, W. S.; D'Souza, W.; Sviridov, D.; Remaley, A. T. Asymmetry in the Lipid Affinity of Bihelical Amphipathic Peptides: A Structural Determinant for the Specificity of ABCA1-Dependent Cholesterol Efflux by Peptides. J. Biol. Chem. 2008, 283, 32273-32282.
51. Tanaka, M.; Takamura, Y.; Kawakami, T.; Aimoto, S.; Saito, H.; Mukai, T. Effect of Amino Acid Distribution of Amphipathic Helical Peptide Derived from Human Apolipoprotein A-1 on Membrane Curvature Sensing. FEBS Lett. 2013, 58⁷, 510-515.
52. Segrest, J. P.; Chung, B. H.; Brouillette, C. G.; Kanellis, P.; McGahan, R. Studies of Synthetic Peptide Analogs of the Amphipathic Helix. Competitive Displacement of Exchangeable Apolipoproteins from Native Lipoproteins. J. Biol. Chem. 1983, 258, 2290-2295.
53. Kingwell, B. A.; Chapman, M. J. Future of High-Density Lipoprotein Infusion Therapies. Circulation 2013, 128, 1112-1121.
54. Murch, O.; Collin, M.; Hinds, C. J.; Thiemermann, C. Lipoproteins in Inflammation and Sepsis. I. Basic Science. Intensive Care Med. 2007, 33, 13-24.
55. Jiao, Y.-l.; Wu, M.-P. Apolipoprotein A-I Diminishes Acute Lung Injury and Sepsis in Mice Induced by Lipoteichoic Acid. Cytokine 2008, 43, 83-87.
56. Zamanian-Daryoush, M.; Lindner, D.; Tallant, T. C.; Wang, Z.; Buffa, J., Klipfell, E.; Parker, Y.; Hatala, D.; Parsons-Wingerter, P.; Rayman, P.; Yusufishaq, M. S. S.; Fisher, E. A.; Smith, J. D.; Finke, J.; DiDonato, J. A.; Hazen, S. L. The Cardioprotective Protein Apolipoprotein A1 Promotes Potent Anti-Tumorigenic Effects. J. Biol. Chem. 2013, 288, 21237-21252.
57. De Nardo, D.; Labzin, L. I.; Kono, H.; Seki, R.; Schmidt, S. V.; Beyer, M.; Xu, D.; Zimmer, S.; Lahrmann, C.; Schildberg, F. A.; Vogelhuber, J.; Kraut, M.; Ulas, T.; Kerksiek, A.; Krebs, W.; Bode, N.; Grebe, A.; Fitzgerald, M. L.; Hernandez, N. J.; Williams, B. R. G.; et al. High-Density Lipoprotein Mediates Anti-Inflammatory Reprogramming of Macrophages via the Transcriptional Regulator ATF3. Nat. Immunol. 2014, 15, 152-160.
58. Catapano, A. L.; Pirillo, A.; Bonacina, F.; Norata, G. D. HDL in Innate and Adaptive Immunity. Cardiovasc. Res. 2014, 103, 372-383.
59. White, C. R.; Garber, D. W.; Anantharamaiah, G. M. Anti-Inflammatory and Cholesterol-Reducing Properties of Apolipoprotein Mimetics: A Review. J. Lipid Res. 2014, 55, 2007-2021.
60. Remaley, A. T.; Amar, M.; Sviridov, D. HDL-Replacement Therapy: Mechanism of Action, Types of Agents and Potential Clinical Indications. Expert Rev. Cardiovasc. Ther. 2008, 6, 1203-1215.
61. Buga, G. M.; Frank, J. S.; Mottino, G. A.; Hakhamian, A.; Narasimha, A.;
Watson, A. D.; Yekta, B.; Navab, M.; Reddy, S. T.; Anantharamaiah, G. M.; Fogelman,
A. M. D-4F Reduces E06 Immunoreactivity, SREBP-1c mRNA Levels, and Renal Inflammation in LDL Receptor-Null Mice Fed a Western Diet. J. Lipid Res. 2008, 49, 192-205.
62. McGrath, K. C.; Li, X.; Twigg, S. M.; Heather, A. K. Apolipoprotein-AI Mimetic Peptides D-4F and L-5F Decrease Hepatic Inflammation and Increase Insulin Sensitivity in C57BL/6 Mice. PLoS One 2020, 15, e0226931.
63. Charles-Schoeman, C.; Banquerigo, M. L.; Hama, S.; Navab, M.; Park, G. S.; Van Lenten, B. J.; Wagner, A. C., Fogelman, A. M.; Brahn, E. Treatment with an Apolipoprotein A-1 Mimetic Peptide in Combination with Pravastatin Inhibits Collagen-Induced Arthritis. Clin. Immunol. 2008, 127, 234-244.
64. Yao, X.; Dai, C.; Fredriksson, K.; Dagur, P. K.; McCoy, J. P.; Qu, X.; Yu, Z.-X.; Keeran, K. J.; Zywicke, G. J.; Amar, M. J. A.; Remaley, A. T.; Levine, S. J. 5A, an Apolipoprotein A-I Mimetic Peptide, Attenuates the Induction of House Dust Mite-Induced Asthma. J. Immunol. 2011, 186, 576-583.
65. Ditiatkovski, M., Palsson, J.; Chin-Dusting, J.; Remaley, A. T.; Sviridov, D. Apolipoprotein A-I Mimetic Peptides. Atertio. Thromb. Vasc. Biol. 2017, 37, 1301-1306.
66. Zhao, Y.; Imura, T.; Leman, L. J.; Curtiss, L. K.; Maryanoff, B. E.; Ghadiri, M. R. Mimicry of High-Density Lipoprotein: Functional Peptide-Lipid Nanoparticles Based on Multivalent Peptide Constructs. J. Am. Chem. Soc. 2013, 135, 13414-13424.
67. Getz, G. S.; Wool, G. D.; Reardon, C. A. HDL Apolipoprotein-Related Peptides in the Treatment of Atherosclerosis and Other Inflammatory Disorders. Curr. Pharm. Des. 2010, 16, 3173-3184.
68. Bloedon, L. T.; Dunbar, R.; Duffy, D.; Pinell-Salles, P.; Norris, R.; DeGroot, B. J.; Movva, R.; Navab, M.; Fogelman, A. M.; Rader, D. J. Safety, Pharmacokinetics, and Pharmacodynamics of Oral ApoA-I Mimetic Peptide D-4F in High-Risk Cardiovascular Patients. J. Lipid Res. 2008, 49, 1344-1352.

### Supporting Information

Full experimental materials and methods; Supplemental Tables S1 and S2 with symmetries, lattice parameters, and NGC values of induced cubic phases; Supplemental FIGS. 6-9 of the SAXS spectra of control samples, SAXS spectra of membranes treated with NGC-generating peptides and APP-2498, schematic of a single peptide interacting with a curved membrane, and schematic of membrane-bound peptides in a model membrane pore (PDF).

### Materials and Methods

### Peptides

Lyophilized histone H4 N-terminus (H4n, S-G-R-G-K-G-G-K-G-L-G-K-G-G-A-K-R-H-R-K-V-L-R-D) (SEQ ID NO: 3) and anti-pore peptide APP-2498 (G-D-A-V-R-E-W-F-E-K-A-W-Q-R-V-R-E-F-F) (SEQ ID NO: 1) synthesized using solid-phase synthesis were purchased at high purity (>95% HPLC) from LifeTein. Lyophilized protegrin-1 (PG-1, R-G-G-R-L-C-Y-C-R-R-R-F-C-V-C-V-G-R) (SEQ ID NO: 4), human immunodeficiency virus type 1 trans-activator of transcription (HIV-TAT, G-R-K-K-R-R-Q-R-R-R-P-Q) (SEQ ID NO: 5), and melittin (G-I-G-A-V-L-K-V-L-T-T-G-L-P-A-L-I-S-W-I-K-R-K-R-Q-Q) (SEQ ID NO: 6) were purchased from Anaspec. Lyophilized apoA-I mimetic peptide apoMP₁ (D-W-F-K-A-F-Y-D-K-V-A-E-K-F-K-E-A-F) (SEQ ID NO: 2) was a generous gift from Srinivasa T. Reddy and Alan M. Fogelman.

### Preparation of SUVs

Lyophilized phospholipids 1,2-dioleoyl-sn-glycero-3-phospho-L-serine (DOPS), 1,2-dioleoyl-sn-glycero-3-phosphoethanolamine (DOPE), and cholesterol (CH) purchased from Avanti Polar Lipids were dissolved in chloroform at 20 mg/mL to produce individual stock solutions. Ternary lipid compositions were prepared from these stock solutions as mixtures of DOPS/DOPE/CH at molar ratios of 20/80/0 and 20/70/10, evaporated under nitrogen, and desiccated overnight under vacuum to form dry lipid films. Lipid films were resuspended in physiological aqueous buffer (140 mM NaCl, 10 mM N-(2-hydroxyethyl)piperazine-N′-ethanesulfonic acid (HEPES), pH 7.4) to a concentration of 20 mg/mL. Lipid suspensions were incubated overnight at 37° C., sonicated until clear, and extruded through a 0.2 μm pore Anotop syringe filter (Whatman) to yield SUVs.

### SAXS Experiments

Before use, peptides were dissolved in nuclease-free water. Depending on the assay, SUVs were then mixed with peptides at specific P/L molar ratios. Precipitated peptide-lipid complexes were transferred into 1.5 mm quartz capillaries (Hilgenberg GmbH, Mark-tubes) and hermetically sealed with an oxygen torch. SAXS measurements were taken at the Stanford Synchrotron Radiation Lightsource (SSRL) (beamline 4-2) and the Advanced Light Source (ALS) (beamline 7.3.3) using monochromatic X-rays with energies of 9 keV and 10 keV, respectively. Samples were incubated at 37° C. and centrifuged before measurement. Scattered radiation was collected using DECTRIS PILATUS3 X 1M and PILATUS 2M detectors (both with a 172 μm pixel size) at SSRL and ALS, respectively. The 21) powder diffraction patterns were azimuthally integrated into 1D patterns using the Nika¹ 1.76 package for Igor Pro 7.04 (Wavemetrics). For all samples, multiple measurements were taken at different times to ensure consistency.

To determine the phases present in each sample, the integrated scattering intensity 1(q) versus q was plotted using Mathematica (Wolfram Research) or MATLAB (MathWorks). The measured q-values corresponding to peak positions were obtained and their ratios were compared to the permitted reflections for different liquid-crystalline lipid phases (e.g., lamellar, hexagonal, cubic). Lamellar phases exhibit integer ratios of 1:2:3 and hexagonal phases exhibit ratios of √1:√3:√4:√7:√9:√12:√13. Cubic phases observed in our experiments belonged to the Pn3m space group, which permits reflections at ratios of √2:√3:√4:√6:√8:√9, and the Im3m space group, which permits reflections at ratios of √2:√4:√6:√8:√10:√12:√14:√16. For each cubic phase, the measured peak positions were related to the Miller indices (h, k, l) of their observed reflections with the equation q=2π√(h²+k²+l²)/a, where a is the lattice parameter. The slope of the linear regression for measured q-values versus √(h²+k²+l²) was then used to calculate a. The average Gaussian curvature (K) per unit cell volume for a cubic phase was calculated using the equation (K)=2πχ/A₀a², where χ is the Euler characteristic and A₀ is the surface area per cubic unit cell. For Pn3m cubic phases, χ=−2 and A₀=1.919. For Im3m cubic phases, χ=−4 and A₀=2.345.

### Schematics of Cubic Phases

The cubic phase representation (FIG. 2c) was generated with Mathematica using the level-set equations.²

### Mouse Procedures and Tissue Processing

All mouse experiments were performed according to European guidelines for the care and use of laboratory animals. Protocols were approved by the Committee on the Ethics of Animal Experiments of the Regierung von Oberbayern.

Animals were housed according to institutional regulations with ad libitum access to food and water. All mice were C57BL/6J background. For the endotoxemia model, female mice (16 weeks old) were challenged with 10 mg/kg LPS from E. coli (0111:B4) and treated with one single dose of 250 μg of apoMP₁ intraperitoneally or saline as control. 24 hours later, mice were euthanized by ketamine/xylacine overdose, retroorbital blood was collected, and the mice were flushed with 20 mL of ice-cold PBS-EDTA (5 mM EDTA). Lung, liver, kidney, and heart were isolated and embedded in Tissue Tek O.C.T. compound (Sakura Finetek) for analysis.

### Immunofluorescence

Cryosections (4 μm) were fixed with cold acetone followed by antigen blockade using 5% goat serum/phosphate buffered saline. Next, sections were incubated overnight at 4° C. with the following primary antibodies: rat anti-mouse Ly6G (BD, 1:200), rabbit anti-mouse histone H4 (Abcam, 1:200), rabbit anti-mouse histone H4 Alexa 488 conjugated (Abcam, 1:200), rabbit anti-mouse citrullinated histone H3 (Abcam, 1:200), rat anti-mouse CD31-Alexa 455 conjugated (BioLegend, 1:50), goat anti-mouse myeloperoxidase (MPO, Millipore, 1:200). After extensive washing, sections were incubated with secondary antibodies conjugated with DyLight 477, Dylight 550, Alexa594, or DyLight 650 (Thermo Fisher, 1:500). Counterstain to visualize nuclei was performed by incubating with DAPI (Molecular Probes). Cell death (TUNEL⁺ cells) was detected using ApopTag® Red in silt Apoptosis Detection Kit (Millipore) following the manufacturer's instructions. Immunofluorescence sections were imaged using a Leica TCS SP8 (Leica Microsystems) equipped with a UV laser, a freely tunable pulsed white light laser, hybrid detectors, and a 63X1.40 oil objective. Raw pictures were deconvolved with Huygens Professional (v.16.10, Scientific Volume Imaging) and maximum intensity projections of deconvolved data were generated with the Leica Application Suite X (v.3.1, Leica Microsystems). Histological sections were quantified by computer-assisted morphometric analysis using ImageJ software (National Institutes of Health).

### Cell Culture and Activation

Mouse vascular aorta/smooth muscle cells (MOVAS) (ATCC, CRL-2797™) were cultured in complete medium (DMEM, Gibco) supplemented with 10% fetal bovine serum (Gibco), 0.2 mg/mL G418 (Invitrogen), and 5 mM sodium pyruvate (Sigma). All cells were maintained in an incubator at 37° C., 5% CO₂. J774A.1 macrophages and SVEC4-10 endothelial cells (both ATCC) were cultured in complete medium (DMEM, Gibco) supplemented with 10% fetal bovine serum (Gibco). HepG2 human hepatocytes were cultured in complete medium (EMEM, Gibco) supplemented with 10% fetal bovine serum (Gibco).

### Cell Viability Assays

Cells were incubated with 50 μg/mL histone H4 (Biomol) and indicated amounts of apoMP₁. Cell viability was measured based on propidium iodide (PI) uptake. PI⁺ cells were visualized using a climate chamber fluorescence microscope (Leica, DMi8) and quantified by ImageJ software.

### Statistics

Statistical analysis was performed by GraphPad Prism 7 (GraphPad Software). The ROUT outlier function was used to exclude statistical outliers (Q=1%). Normal distribution of the data was assessed using the D'Agostino-Pearson omnibus test for normality. Normally distributed data was tested by two-tailed unpaired t-test (one variable) or one-way ANOVA with Tukey's or Dunnet's correction (>2 variables). When two factors were analyzed, data was analyzed using two-way ANOVA with Tukey's correction. In all tests a 95% confidence interval was used, with which p<0.05 was considered a significant difference. Correlograms were generated using R. All data is represented as mean±SEM.

### Modeling Peptide-Lipid Interactions for Pore Formers and Pore Inhibitors

The central hypothesis in this work is that there are peptides that can induce membrane curvatures to form pores in the membrane (ex: H4n induction of NGC (K<0)), as well as peptides that can induce the “opposite” curvature, thereby potentially canceling the membrane remodeling activity of the former and inhibit pore formation (ex: apoMP₁ induction of PGC (K>0)). We develop a theoretical model based on linear membrane elasticity to connect the structural tendency identified by SAXS to pore formation in membranes when combinations of both peptide types are present.

Single Peptide Interacting with a Membrane

Our approach closely follows previous modeling of anisotropic, curvature-inducing proteins or peptides.³⁻⁵ We assume the peptides couple to the local curvature of an isolated 2D membrane. Since NGC is inherently anisotropic, a single peptide induces not just a spontaneous mean curvature in the membrane, but also a spontaneous curvature tensor hᵢⱼ^{±}(θ), whose eigenvectors and eigenvalues give the directions and magnitudes of the largest and smallest membrane curvature induced by the peptide. The sign of the superscript denotes whether the peptide induces PGC or NGC, with

$${h}_{ij}^{\pm } ( θ ) = ( \begin{matrix}cos θ & - sin θ \\ sin θ & cos θ\end{matrix} ) ( \begin{matrix}{ξ}_{1}^{\pm } & 0 \\ 0 & {ξ}_{2}^{\pm }\end{matrix} ) ( \begin{matrix}cos θ & sin θ \\ - sin θ & cos θ\end{matrix} ) , \qquad ( 1 )$$

where ξ₁^{±} and ξ₂^{±} are the peptide-generated curvatures along the principal axes, and the angle θ describes the local orientation of the peptide with respect to a set of surface coordinates (x₁, x₂). For PGC, we assume ξ₁^{±}=ξ₂^{±}, whereas NGC has ξ₁⁻>0 but ξ₂⁻<0. We also define the vector function X(x₁, x₂) to describe the shape of the membrane neutral surface, a unit vector N(x₁, x₂) for the normal to the surface, and a local membrane curvature tensor hᵢⱼ=N·∂ᵢ∂ⱼX, where ∂ᵢ denotes a partial derivative along the xᵢ direction. The mean curvature of the membrane is the trace of hᵢⱼ(x₁, x₂) while the Gaussian curvature is its determinant. Since we expect the interaction energy to be minimized when the membrane curvature matches the spontaneous curvature induced by the peptide, we consider an interaction of the form

$${E}_{\pm } ( θ ; {x}_{1} , {x}_{2} ) = {( k / 2 ) [ {h}_{ij} ( {x}_{1} , {x}_{2} ) - {h}_{ij}^{\pm } ( θ ) ]}^{2} . \qquad ( 2 )$$

We assume the coupling k is the same for both peptide classes. Denoting the monolayer thickness as t, we define a dimensionless coupling constant λ=k/(k_{B}Tt²) at temperature T (Boltzmann constant k_{B}) and rescale all curvatures by 1/t. Being proportional to the interaction energy between a peptide and a flat membrane, λk_{B}T therefore provides a measure of how strongly the peptide couples to the membrane shape. Let c₁ and c₂ denote the maximum and minimum principal curvatures of the membrane respectively, measured in units of t, at (x₁, x₂). Expanding the tensor notation, the energy of a single peptide interacting with the membrane through Eq. (1) becomes³⁻⁵

$${E}_{\pm } ( θ ; {c}_{1} , {c}_{2} ) / {k}_{B} T = \frac{λ}{2} ( [ {( {c}_{1} - {ξ}_{1}^{\pm } )}^{2} + {( {c}_{2} - {ξ}_{2}^{\pm } )}^{2} ] {cos}^{2} θ + [ {( {c}_{2} - {ξ}_{1}^{\pm } )}^{2} + {( {c}_{1} - {ξ}_{2}^{\pm } )}^{2} ] {sin}^{2} θ ) . \qquad ( 3 )$$

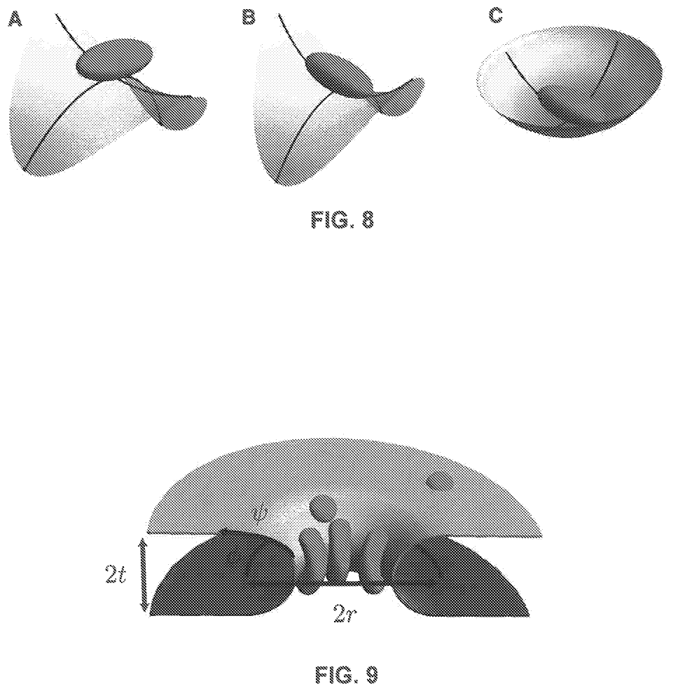

This energy explicitly depends on the coordinates since the principal curvatures cᵢ are functions of the coordinates themselves. When θ=0, the peptide's maximal curvature is aligned with the membrane's and only the first term persists. On the other hand, if θ=π/2, the peptide is rotated by ninety degrees and the peptide's maximal curvature is further penalized by being aligned with the membrane's smallest curvature (FIG. 8a,b). The tensor notation accounts for intermediate-case angles in a concise format. Finally,

$${E}_{\pm }^{F} / ( {k}_{B} T ) = {E}_{\pm } ( θ ; 0 , 0 ) / ( {k}_{B} T ) = \frac{λ}{2} [ {ξ}_{1 , \pm }^{2} + {ξ}_{2 , \pm }^{2} ] \qquad ( 4 )$$

represents the energy of a peptide in a locally flat region of the membrane, and is independent of both angle and coordinates.
Multiple Peptide Types (NGC-Inducing and PGC-Inducing) Interacting with a Membrane

We assume that bound peptide complexes can effectively function as a single unified peptide, and that such complexes do not strongly interact with one another, except by excluding other peptides from binding to the membrane in their immediate vicinity. To account for a large number of peptides, we must statistically average over the location and orientation of each peptide. For reversible peptide-membrane binding, we assume peptides in solution are in equilibrium with bound peptides, so each species is characterized by a chemical potential k_{B}Tln(n_{±}), where n_{±} is the volume fraction of each peptide species in solution, and a membrane binding energy e_{±}^{B}. We define μ_{±}=e_{±}^{B}+k_{B}Tln(n_{±}) to account for both. We also assume different species subtend characteristic areas that are roughly identical—a reasonable approximation given the two prototypical peptides being compared are relatively similar in mass and peptide structure. To account for the excluded area of peptides on the membrane, we decompose the membrane area into sites of area A₀, which can either be occupied or unoccupied with only one peptide type. The Grand partition function for the peptides is found by summing (or integrating) over all possible peptide orientations, positions, and numbers binding to the membrane. This yields

$$\begin{matrix}Q = {\prod }_{sites} ( \int \frac{d θ}{2 π} {e}^{β {μ}_{+} - β {E}_{+} ( θ ; {c}_{1} , {c}_{2} )} + \int \frac{d θ}{2 π} {e}^{β {μ}_{-} - β {E}_{-} ( θ ; {c}_{1} , {c}_{2} )} ) \\ = exp [ \int \frac{dA}{{A}_{0}} ln ( 1 + \int \frac{d θ}{2 π} {e}^{β {μ}_{+} - β {E}_{+} ( θ ; {c}_{1} , {c}_{2} )} + \int \frac{d θ}{2 π} {e}^{β {μ}_{-} - β {E}_{-} ( θ ; {c}_{1} , {c}_{2} )} ) ] ,\end{matrix} \qquad ( 5 )$$

where β=1/k_{B}T and we have taken the continuum limit in the product over the membrane area in the last equality, and ∫dA denotes the integral over the membrane neutral surface. The correction to the Helfrich membrane energy is given by ΔF=−k_{B}TlnQ, so that

$$( 6 )$$

$$\frac{Δ F}{{k}_{B} T} = - \frac{1}{{A}_{0}} \int dA ln ( 1 + \int \frac{d θ}{2 π} {e}^{β {μ}_{+} - β {E}_{+} ( θ ; {c}_{1} , {c}_{2} )} + \int \frac{d θ}{2 π} {e}^{β {μ}_{-} - β {E}_{-} ( θ ; {c}_{1} , {c}_{2} )} ) .$$

The two integrals over angles compute the statistical average over the orientations of the peptides at each site on the membrane, weighted by the energy. The integrand acquires position dependence from the variation of the membrane principal curvatures cᵢ from point to point.

To separate the contribution of the peptides to the membrane surface tension, we add and subtract the change in free energy for a peptide on a flat surface to obtain

$$\frac{Δ F}{{k}_{B} T} = \frac{A}{{A}_{0}} ln ( 1 + {e}^{β {μ}_{+} - β {E}_{+}^{F}} {e}^{β {μ}_{-} - β {E}_{-}^{F}} ) - \frac{1}{{A}_{0}} \int dA [ ln ( 1 + \int \frac{d θ}{2 π} \text{?} + \int \frac{d θ}{2 π} {e}^{β {μ}_{-} - β {E}_{-} ( θ ; {c}_{1} , {c}_{2} )} ) - ln ( 1 + {e}^{β {μ}_{+} - β {E}_{+}^{F}} + {e}^{β {μ}_{-} - β {E}_{-}^{F}} ) ] . \qquad ( 7 )$$

$$\text{?} \text{indicates text missing or illegible when filed}$$

The second term explicitly vanishes on a flat membrane, so a Taylor expansion in powers of membrane curvature will have no zeroth order term. It gives the entire correction to the Helfrich bending energy of a membrane, accounting for the energetics of interaction between peptide and membrane as well as the orientational and translational entropy of the peptides. The first term of Eq. (7) is proportional to area but not curvature, and captures the entire peptide contribution to the membrane surface tension. For a membrane in equilibrium with a lipid reservoir, it accounts for the change in membrane area due to the peptide binding. For example, if binding is highly favorable, the surface tension will decrease and the membrane area will correspondingly increase.

The second term can be better understood by expanding Eq. (7) in powers of membrane curvature. Schematically, this yields ΔF/k_{B}T=−AΔσ+c₀(c₁+c₂)+Δκ(c₁+c₂)²/2+Δκ̅c₁c₂+ . . . , where the correction to the surface tension from the first term is denoted Δσ, and expressions for the spontaneous curvature co and bending moduli corrections, Δκ and Δκ̅, can be obtained but are very cumbersome. To summarize, the peptides act to change the effective bending moduli of the Helfrich model as well as induce a spontaneous curvature.

The integrals over θ can be obtained explicitly, yielding

$$\frac{Δ F}{{k}_{B} T} = \frac{A}{{A}_{0}} ln [ 1 + {\sum }_{\pm } {z}_{\pm } ] - \int \frac{dA}{{A}_{0}} ln \{ 1 + {\sum }_{\pm } {z}_{\pm } exp [ - \frac{λ}{2} ( {c}_{1}^{2} + {c}_{2}^{2} - ( {c}_{1} + {c}_{2} ) ( {ξ}_{1 , \pm } + {ξ}_{2 , \pm } ) ) ] \text{} {I}_{0} [ \text{} \frac{λ}{2} \text{} ( {c}_{1} - {c}_{2} ) ( {ξ}_{1 , \pm } + {ξ}_{2 , \pm } ) ] - ln [ 1 + {\sum }_{\pm } {z}_{\pm } ] \} , \qquad ( 8 )$$

where I₀(x)≥1 is a modified Bessel function and is defined to be symmetric about x=0, and z_{±}=exp[μ_{±}/(k_{B}T)−λ(ξ_{1,±}²+ξ_{2,±}²)/2] is a generalized fugacity for the peptides. Finally, we note the expressions obtained for the peptide area fraction of either type:

$$( 9 )$$

$${ρ}_{\pm } = {𝒩}^{- 1} {z}_{\pm } exp [ - \frac{λ}{2} ( {c}_{1}^{2} + {c}_{2}^{2} - ( {c}_{1} + {c}_{2} ) ( {ξ}_{1 , \pm } + {ξ}_{2 , \pm } ) ) ] {I}_{0} [ \frac{λ}{2} ( {c}_{1} - {c}_{2} ) ( {ξ}_{1 , \pm } - {ξ}_{2 , \pm } ) ] ,$$

where the normalization [symbol] is given by

$$𝒩 = 1 + {\sum }_{\pm } {z}_{\pm } exp [ - \frac{λ}{2} ( {c}_{1}^{2} + {c}_{2}^{2} - ( {c}_{1} + {c}_{2} ) ( {ξ}_{1 , \pm } + {ξ}_{2 , \pm } ) ) ] {I}_{0} [ \frac{λ}{2} ( {ξ}_{1 , \pm } - {ξ}_{2 , \pm } ) ] . \qquad ( 10 )$$

The quantities z_{±} can be interpreted by considering the area fraction on a flat membrane, for which ρ_{±}^{F}=z_{±}/(1+z₊+z₋). Thus, the generalized fugacities are related to the average density of peptides on the flat regions of the membrane. Similarly, we can interpret the quantity A by considering the additional cost of inserting a peptide on a flat membrane region rather than one for which the induced curvature is precisely matched by the membrane (see Eq. (4)).

### Modeling of Membrane Pores

We describe a pore with the fixed shape of the inside of a torus (FIG. 9), having X=[r+cos Ψ][cos ϕ\hat{x}+sin ϕŷ]+sin Ψ\hat{z}, measured in units of t, for π/2≤Ψ≤3π/2, 0≤ϕ≤2π and pore radius r≥1. This yields principal curvatures c₁=1 and c₂=cos Ψ/(r+cos Ψ). The area measure is given by dA=(r+cos) d_{Ψ}d_{ϕ}. In the absence of peptides, pore formation is typically described using a model reminiscent of classical nucleation theory,^{6, 7} with F_{bare}/(k_{B}T)=−σπ²+2πΓ for a circular pore of radius r with line tension Γ and a membrane surface tension σ. This free energy describes a barrier height F_{barrier}=πk_{B}T Γ²/σ. As we saw earlier from Eq. (7), the presence of peptides modifies the bare line and surface tensions.

For given peptide fugacities and pore radii, we compute the free energy over the inner rim of a pore numerically using Eq. (8). We assume a bare line tension Γ of around 10⁻¹¹ J/m, so using t≈2 nm gives Γt≈5. Similarly, a typical surface tension is 10⁻³ N/m², which gives σt²≈1. We further assume A₀≈t² and λ=7.5 which corresponds to an energy difference of ˜4 k_{B}T between a peptide bound to a flat membrane and one bound at a point at which the membrane exactly matches the prescribed curvature of the peptide. Finally, we choose prescribed curvatures for the peptides by using the characteristic length scale of the bicontinuous cubic phases determined by X-ray scattering (FIG. 2d). For K<0-inducing (−) peptides, we set ξ_{1,-}=1/t≈0.5 nm⁻¹ commensurate with the curvature on the inside of a pore and (λ/2)(ξ₁²+ξ₂²)≈4k_{B}T to give an average induced Gaussian membrane curvature of [symbol] K [symbol] ≈0.3/t)² nm⁻², consistent with a Schwarz' D (Pn3m) surface of lattice size 17 nm.^{8, 9} A K>0-inducing (+) peptide may induce different amounts of positive curvature stress along its two principal directions, but we adopt a simple model of isotropic curvature presentation by using ξ_{1,+}=ξ_{2,+}=0.3/t nm⁻¹. FIG. 4a plots the energy barrier for opening a membrane pore in a mixture of K>0-inducing (+) peptides (orange) and K<0-inducing (−) peptides (green). Even at very modest area fractions, a K>0-inducing (+) peptide will inhibit pore formation by raising the energy barrier drastically. We find that K<0-inducing (−) peptides, like H4n, suppress the free energy barrier while K>0-inducing (+) peptides, like apoMP₁, elevate it. Conversely, equilibrium Pn3m lattice constants a_{Pn3m} can be estimated by minimizing the free energy for given peptide-prescribed 51.2 and H4n/apoMP₁ stoichiometry. We minimized the sum of the corrections from Eq. (8) and the Helfrich energy density

$$\frac{ε}{{k}_{B} T} = \frac{κ}{2} {( {c}_{1} + {c}_{2} )}^{2} + \overline{κ} {c}_{1} {c}_{2} , \qquad ( 11 )$$

using literature values for the moduli κ̅=−0.83κ and κ/(k_{B}T)=10.¹⁰ See FIG. 4b for computed a_{Pn3m} versus peptide stoichiometry. For the K<0-inducing (−) peptide, peptide-prescribed curvatures were kept at (ξ₁, ξ₂)=(1,−0.09), in units of 1/t, for maximum compatibility with a 17 nm Pn3m phase. For the K>0-inducing (+) peptide, we varied curvatures isotropically (ξ₁=ξ₂) from 0.3 to 0.74, in units of 1/t. Consistent with experimental trends, calculated Pn3m lattice constants generally grow with increasing molar ratio of K>0-generating (apoMP₁) to K<0-generating (H4n) peptides.

It is interesting to note that experimentally, upon addition of PGC-generating molecules to cubic phases induced by NGC-generating molecules, we see first a small initial decrease in the cubic lattice constant, followed by a sustained increase in the cubic lattice constant, followed finally by a complete suppression of the cubic phase. (In other PGC/NGC systems, we have also seen the lattice constant of the cubic phase increase monotonically until the cubic phase is completely suppressed.) We rationalize this interesting behavior in the following manner. By definition, a cubic phase is characterized by zero mean curvature, which requires the quantitative amount of positive curvature to be the same as that of negative curvature in the system. However, for an arbitrary membrane-remodeling molecule that induces NGC, the amount of induced positive curvature is in general not necessarily the same as the amount of induced negative curvature. For example, if a molecule induces more negative curvature than positive curvature, a cubic phase can in principle still form at a different lattice constant at the expense of additional membrane stress. If one adds more PGC-inducing molecules to this system, it may allow the system to form optimal cubic phases with balanced positive and negative curvatures and thereby initially decrease the lattice constant from the stressed value, before so much PGC is added that the cubic phase itself is destabilized.

### Supplemental Tables

TABLE S1
The symmetries, lattice parameters, and NGC values of cubic phases
induced by histone H4n (PS/PE/CH 20/70/10), HIV-TAT (PS/PE 20/80),
and protegrin-1 (PS/PE 20/80) in the absence and presence of apoMP₁.

| ApoMP₁ | Histone H4n | HIV-TAT | ApoMP₁ | Protegrin-1 |
|---|---|---|---|---|
| P/L molar | (P/L molar | (P/L molar | P/L molar | (P/L molar |
| ratio | ratio = 1/40) | ratio = 1/40) | ratio | ratio = 1/40) |
| 0 | Pn3m (α = 18.3 nm) | Pn3m (α = 19.6 nm) | 0 | Im3m (α = 18.0 nm) |
| | <K> = −1.96E−2 nm⁻² | <K> = −1.70E−2 nm⁻² | | <K> = −3.3E−2 nm⁻² |
| | | | | Pn3m (α = 14.5 nm) |
| | | | | <K> = −3.13E−2 nm⁻² |
| 1/100 | Pn3m (α = 18.2 nm) | Pn3m (α = 17.0 nm) | 1/120 | Pn3m (α = 18.4 nm) |
| | <K> = −1.98E−2 nm⁻² | <K> = −2.26E−2 nm⁻² | | <K> = −1.94E−2 nm⁻² |
| 1/50 | Pn3m (α = 17.2 nm) | Pn3m (α = 15.3 nm) | 1/60 | |
| | <K> = −2.22E−2 nm⁻² | <K> = −2.79E−2 nm⁻² | | |
| 1/25 | | | 1/30 | |

*Empty cells indicate the absence of cubic phases.

TABLE S2
The symmetries, lattice parameters, and NGC values of cubic phases
induced by histone H4n (PS/PE/CH 20/70/10), melittin (PS/PE 20/80),
and HIV-TAT (PS/PE 20/80) in the absence and presence of APP-2498.

| APP-2498 | Histone H4n | Melittin | HIV-TAT |
|---|---|---|---|
| P/L molar | (P/L molar | (P/L molar | (P/L molar |
| ratio | ratio = 1/40) | ratio = 1/25) | ratio = 1/40) |
| 0 | Pn3m (α = 18.3 nm) | Im3m (α = 24.0 nm) | Pn3m (α = 19.6 nm) |
| | <K> = −1.96E−2 nm⁻² | <K> = −1.86E−2 nm⁻² | <K> = −1.70E−2 nm⁻² |
| 1/100 | Pn3m (α = 17.6 nm) | Im3m (α = 17.5 nm) | Pn3m (α = 20.0 nm) |
| | <K> = −2.11E−2 nm⁻² | <K> = −3.48E−2 nm⁻² | <K> = −1.64E−2 nm⁻² |
| 1/50 | Pn3m (α = 16.5 nm) | | Pn3m (α = 18.0 nm) |
| | <K> = −2.39E−2 nm⁻² | | <K> = −2.02E−2 nm⁻² |
| 1/25 | | | |

*Empty cells indicate the absence of cubic phases.
*Empty cells indicate the absence of cubic phases.

### SUPPLEMENTAL MATERIAL REFERENCES

1. Ilavsky, J. Nika: Software for Two-Dimensional Data Reduction. J. Appl. Crystallogr. 2012, 45, 324-328.
2. Wohlgemuth, M.; Yufa, N.; Hoffman, J.; Thomas, E. L. Triply Periodic Bicontinuous Cubic Microdomain Morphologies by Symmetries. Macromolecules 2001, 34, 6083-6089.
3. Fournier, J. B. Nontopological Saddle-Splay and Curvature Instabilities from Anisotropic Membrane Inclusions. Phys. Rev. Lett. 1996, 76, 4436-4439.
4. Kralj-Iglič, V.; Heinrich, V.; Svetina, S.; Žekš, B. Free Energy of Closed Membrane with Anisotropic Inclusions. Eur. Phys. J. B 1999, 10, 5-8.
5. Fošnarič, M.; Kralj-Iglič, V.; Bohinc, K.; Iglič, A.; May, S. Stabilization of Pores in Lipid Bilayers by Anisotropic Inclusions. J. Phys. Chem. B 2003, 107, 12519-12526.
6. Litster, J. D. Stability of Lipid Bilayers and Red Blood Cell Membranes. Phys. Lett. A 1975, 53, 193-194.
7. García-Sáez, A. J.; Chiantia, S.; Salgado, J., Schwille, P. Pore Formation by a Bax-Derived Peptide: Effect on the Line Tension of the Membrane Probed by AFM. Biophys. J. 2007, 93, 103-112.
8. Shearman, G. C.; Ces, O.; Templer, R. H.; Seddon, J. M. Inverse Lyotropic Phases of Lipids and Membrane Curvature. J. Phys.: Condens. Matter 2006, 18, S1105-S1124.
9. Lord, E. A.; Mackay, A. L. Periodic Minimal Surfaces of Cubic Symmetry. Curr. Sci. 2003, 85, 346-362.
10. Siegel, D. P.; Kozlov, M. M. The Gaussian Curvature Elastic Modulus of N-Monomethylated Dioleoylphosphatidylethanolamine: Relevance to Membrane Fusion and Lipid Phase Behavior. Biophys. J. 2004, 87, 366-374.
11. Matsuzaki, K.; Yoneyama, S.; Miyajima, K. Pore Formation and Translocation of Melittin. Biophys. J. 1997, 73, 831-838.

### REFERENCES

All publications mentioned herein (e.g., the references numerically listed above; and Lee et al., ACS Nano 2021, 15, 15930-15939; Dathe et al., Biochimica et Biophysica Acta 1462 (1999) 71-87; and Dathe et al., Biochimica et Biophysica Acta 1558 (2002) 171-186) are incorporated herein by reference to disclose and describe aspects, methods and/or materials in connection with the cited publications. The inventor's lab website and publication page can be accessed by searching “/wonglab.seas.ucla.edu/” and “/wonglab.seas.ucla.edu/publications/”.

1. A process for making a peptide product, the process comprising forming a peptide comprising a helix from a plurality of amino acids selected such that when visualized in a helical wheel plot diagram depicting projections of amino acid residue positions onto a plane that is perpendicular to the axis of the helix of the peptide:
the peptide sequence comprises a plurality of amino acid residues selected to forms three segments of amino acids in the peptide product, wherein the three segments of amino acids comprise: a nonpolar segment of amino acid residues, a polar segment of amino acid residues, and a segment of positively charged amino acid residues such that:
(1) the nonpolar segment comprises 6-11 continuous amino acid residues which together subtend a radial angle of 120-220° perpendicular to the axis of the helix; and the nonpolar segment comprises at least 4 amino acid residues selected from A, C, F, G, I, L, M, P, V, W and Y;
(2) the polar segment comprises 4-13 continuous amino acid residues which together subtend a radial angle of 80-260° perpendicular to the axis of the helix; and the polar segment comprises at least 4 amino acid residues selected from A, C, D, E, G, H, K, N, P, Q, R, S, T and Y; and
(3) the positively charged segment comprises 1-6 continuous amino acid residues which together subtend a radial angle of 20-120° perpendicular to the axis of the helix; and the positively charged segment comprises at least 1 amino acid residue selected from H, K and R;
and forming the peptide product from the plurality of selected amino acids residues so that the peptide product is made.

2. The process of claim 1, wherein the peptide product is further selected for an ability to induce positive Gaussian curvature in a mammalian cell contacted with the peptide product and/or an ability to inhibit negative Gaussian curvature in a mammalian cell contacted with the peptide product.

3. The process of claim 1, wherein:
the peptide product comprises least one amino residue selected from: G, N, Q and S;
the peptide product does not comprise more than 5 aromatic nonpolar amino acid residues; and/or
the peptide product exhibits a net charge between −3 and +3 at physiological pH.

4. The process of claim 1, further comprising coupling the peptide product to a plurality of amino acids so as to form a polypeptide.

5. The process of claim 4, wherein the peptide is coupled to a plurality of amino acids that form a polypeptide sequence expressed by a mammalian genome.

6. The process of claim 1, a peptide product does not have the sequence: D-W-F-K-A-F-Y-D-K-V-A-E-K-F-K-E-A-F.

7. The process of claim 1, wherein the peptide comprises the sequence:
G-D-A-V-R-E-W-F-E-K-A-W-Q-R-V-R-E-F-F.

8. The process of claim, further comprising combining the peptide with a pharmaceutically acceptable carrier.

9. A peptide product made by the process of claim 1.

10. The peptide of claim 9, wherein the peptide induces positive Gaussian curvature in a mammalian cell contacted with the peptide product and/or counteracts negative Gaussian curvature in a mammalian cell contacted with the peptide product.

11. A composition of matter comprising a polynucleotide encoding a peptide made by the process of claim 1.

12. The composition of claim 11, wherein the peptide comprises the sequence: G-D-A-V-R-E-W-F-E-K-A-W-Q-R-V-R-E-F-F.

13. The composition of claim 11, wherein the polynucleotide encoding the peptide is fused in frame to a polynucleotide sequence encoding a plurality of amino acids.

14. The composition of claim 11, wherein the polynucleotide encoding the peptide is disposed within a vector comprising sequences for expressing the peptide in a mammalian cell.

15. A method of inducing positive Gaussian curvature in a mammalian cell and/or counteracting negative Gaussian curvature in a mammalian cell, the method comprising contacting the mammalian cell with a composition comprising a peptide of claim 9, wherein concentrations of the peptide in the composition are selected to be sufficient to induce positive Gaussian curvature in the mammalian cell and/or counteract negative Gaussian curvature in the mammalian cell when the mammalian cell is contacted with the composition.

16. The method of claim 15, wherein the method is adapted to prevent fertilization by inhibiting membrane fusion between sperm and egg cells.

17. The method of claim 15, wherein the method is adapted to inhibit viral replication processes requiring negative Gaussian membrane curvature.

18. The method of claim 15, wherein the method is adapted to mitigate virus-associated inflammation.

19. The method of claim 15, wherein the method is adapted to mitigate inflammation resulting from lytic cell death or tissue damage by inhibiting permeabilization of cell membranes.

20. The method of claim 15, wherein the method is adapted to:
interfere with and/or inhibit membrane fusion required for secretory processes, including, but not limited to, neurotransmitter release, hormone secretion, and enzyme release; and/or
stabilize natural or artificial lipid membranes so as to inhibit said membrane permeabilization.


SEQUENCE LISTING (18 sequences)

SEQ ID NO: 1 - protein, 19 aa, synthetic construct
GDAVREWFEKAWQRVREFF

SEQ ID NO: 2 - protein, 18 aa, synthetic construct
DWFKAFYDKVAEKFKEAF

SEQ ID NO: 3 - protein, 24 aa, synthetic construct
SGRGKGGKGLGKGGAKRHRKVLRD

SEQ ID NO: 4 - protein, 18 aa, synthetic construct
RGGRLCYCRRRFCVCVGR

SEQ ID NO: 5 - protein, 12 aa, synthetic construct
GRKKRRQRRRPQ

SEQ ID NO: 6 - protein, 26 aa, synthetic construct
GIGAVLKVLTTGLPALISWIKRKRQQ

SEQ ID NO: 7 - protein, 19 aa, synthetic construct
FFERVRQWAKEFWERVADG

SEQ ID NO: 8 - protein, 19 aa, synthetic construct
GDAVREVIEKAVQRVREIV

SEQ ID NO: 9 - protein, 19 aa, synthetic construct
GDAVKEWFEKAWQKVKEFF

SEQ ID NO: 10 - protein, 19 aa, synthetic construct
GERAKEWVEAFWEKAREYF

SEQ ID NO: 11 - protein, 19 aa, synthetic construct
GERVKEFFEAFFEKAREYW

SEQ ID NO: 12 - protein, 19 aa, synthetic construct
GEKAKEWVQAFWQKAKEYF

SEQ ID NO: 13 - protein, 19 aa, synthetic construct
GEKVKEFFQAFFQKAKEYW

SEQ ID NO: 14 - protein, 19 aa, synthetic construct
GDAVKEWFEKAWQKVKEFL

SEQ ID NO: 15 - protein, 19 aa, synthetic construct
GEQLKQKFQEFWDKLKEWY

SEQ ID NO: 16 - protein, 19 aa, synthetic construct
GEKLKQKAQEFFDAVKEWF

SEQ ID NO: 17 - protein, 21 aa, synthetic construct
GKEKAEEFFQALKEWFDKFKN

SEQ ID NO: 18 - protein, 19 aa, synthetic construct
GEQFKQAFQEWWDKLKEKY